(19) United States
(12) Patent Application Publication
(10) Pub. No.: US 2007/0270475 A1
(43) Pub. Date: Nov. 22, 2007

(54) 4-Methoxy-Pyrrolidine-2-Carboxylic Acid Compounds and Derivatives Thereof as Hepatitis C Virus Inhibitors

(75) Inventors: Rossella Guidetti, c/o Stevenage (GB); David Haigh, Stevenage (GB); Charles David Hartley, Stevenage (GB); Peter David Howes, Stevenage (GB); Fabrizio Nerozzi, Stevenage (GB); Stephen Allan Smith, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford, Middlesex (GB)

(21) Appl. No.: 11/568,127

(22) PCT Filed: Oct. 24, 2005
(86) PCT No.: PCT/EP05/11532

§ 371 Date: Oct. 20, 2006

(30) Foreign Application Priority Data
Oct. 25, 2004 (GB) 0423673.3

(51) Int. Cl. C07D 207/04; C07D 277/02

(52) U.S. Cl. 514/365; 514/403; 514/422; 548/204; 548/364.1; 548/571

(57) ABSTRACT

Anti-viral agents of Formula (Ia) 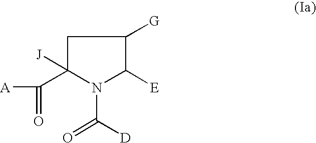 wherein: A represents hydroxy; D represents 4-tert-butyl-3-methoxyphenyl; E represents 1,3-thiazol-2-yl or 5-methyl-1,3-thiazol-2-yl,; G represents methoxymethyl; J represents 1,3-thiazol-2-ylmethyl, 1,3-thiazol-4-ylmethyl, 1,2-thiazol-3-ylmethyl, or 1H-pyrazol-1-ylmethyl; and salts, solvates and esters thereof; provided that when A is esterified to form —OR where R is selected from straight or branched chain alkyl, aralkyl, aryloxyalkyl, or aryl, then R is other than tert-butyl; processes for their preparation and their use in HCV treatment are provided.

### FIELD OF THE INVENTION

The present invention relates to novel C(4)-methoxymethyl acyl pyrrolidine derivatives useful as anti-viral agents. Specifically, the present invention involves novel Hepatitis C Virus (HCV) inhibitors.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang CY et al ‘An RNA pseudoknot is an essential structural element of the internal ribosome entry site located within the hepatitis C virus 5′ noncoding region’ RNA-A Publication of the RNA Society. 1(5): 526-537, 1995 Jul.). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p931-960; Raven Press, N.Y.). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S.E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across lb isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al. (2000) Journal of Virology, 74(4), p. 2046-2051). Thus, inhibition of NS5B RdRp activity (inhibition of RNA replication) is predicted to cure HCV infection.

Although the predominant HCV genotype worldwide is genotype 1, this itself has two main subtypes, denoted 1a and 1b. As seen from entries into the Los Alamos HCV database (www.hcv.lanl.gov) (Table 1) there are regional differences in the distribution of these subtypes: while genotype 1a is most abundant in the United States, the majority of sequences in Europe and Japan are from genotype 1b.

TABLE 1

| % of sequences | | | | |
|---|---|---|---|---|
| in the database | World | USA | Europe | Japan |
| Genotype 1 | 71.8 | 87.8 | 75.9 | 80.2 |
| Genotype 1a | 28.4 | 66.4 | 21.7 | 1.6 |
| Genotype 1b | 43.4 | 21.4 | 54.2 | 78.6 |

The prevalance of genotype la in some regions makes it highly desirable to identify an anti-viral agent that is able to inhibit both genotype 1a and genotype 1b. This means a wider patient pool would be able to benefit from treatment with the same agent.

Based on the foregoing, there exists a significant need to identify synthetic or biological compounds for their ability to inhibit replication of both genotype la and genotype lb of HCV.

PCT publication number WO2004/037818 generically discloses certain compounds, including certain acyl pyrrolidine compounds, having HCV inhibitory activity. The assay is directed to the 1b genotype. The compounds disclosed have the formula (I)

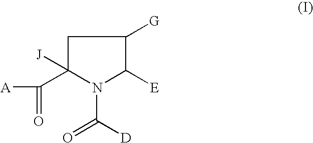

wherein:

A represents hydroxy;

D represents aryl or heteroaryl;

E represents hydrogen, C₁₋₆alkyl, aryl, heteroaryl or heterocyclyl;

G represents hydrogen or C₁₋₆alkyl optionally substituted by one or more substituents selected from halo, OR¹, SR¹, C(O)NR²R³, CO₂H, C(O)R⁴, CO₂R⁴, NR²R³, NHC(O)R⁴, NHCO₂R⁴, NHC(O)NR⁵R⁶, SO₂NR⁵R⁶, SO₂R⁴, nitro, cyano, aryl, heteroaryl and heterocyclyl;

R¹ represents hydrogen, C₁₋₆alkyl, arylalkyl, or heteroarylalkyl;

R² and R³ are independently selected from hydrogen, C₁₋₆-alkyl, aryl and heteroaryl; or R² and R³ together with the nitrogen atom to which they are attached form a 5 or 6 membered saturated cyclic group;

R⁴ is selected from the group consisting of C₁₋₆alkyl, aryl, heteroaryl, arylalkyl, and heteroarylalkyl;

R⁵ and R⁶ are independently selected from the group consisting of hydrogen, C₁₋₆alkyl, aryl, heteroaryl, arylalkyl, and heteroarylalkyl; or R⁵ and R⁵ together with the nitrogen atom to which they are attached form a 5 or 6 membered saturated cyclic group; and J represents C₁₋₆alkyl, heterocyclylalkyl, arylalkyl or heteroarylalkyl; and salts, solvates and esters thereof; provided that when A is esterified to form —OR where R is selected from straight or branched chain alkyl, aralkyl, aryloxyalkyl, or aryl, then R is other than tert-butyl.

Surprisingly, it has now been found that compounds according to the present invention, generically disclosed in WO2004/037818, and having a specific substitution pattern, exhibit improved properties over those compounds specifically disclosed in WO2004/037818.

### SUMMARY OF THE INVENTION

The present invention involves C(2′)-heteroarylmethyl-C(4)-methoxymethyl acyl pyrrolidine compounds represented hereinbelow, pharmaceutical compositions comprising such compounds and use of the compounds in treating viral infection, especially HCV infection.

### DETAILED DESCRIPTION OF THE INVENTION

The present invention provides at least one chemical entity chosen from compounds of Formula (Ia):

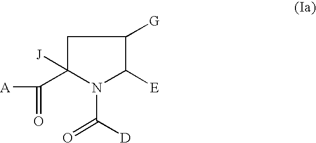

wherein:

A represents hydroxy;

D represents 4-tert-butyl-3-methoxyphenyl;

E represents 1,3-thiazol-2-yl or 5-methyl-1,3-thiazol-2-yl;

G represents methoxymethyl;

J represents 1,3-thiazol-2-ylmethyl, 1,3-thiazol-4-ylmethyl, 1,2-thiazol-3-ylmethyl, or 1H-pyrazol-1-ylmethyl;

and salts, solvates and esters thereof; provided that when A is esterified to form —OR where R is selected from straight or branched chain alkyl, aralkyl, aryloxyalkyl, or aryl, then R is other than tert-butyl.

In one aspect, the relative stereochemistry of racemic compounds of Formula (Ia), is represented by Formulae (Ip) or (Iq): 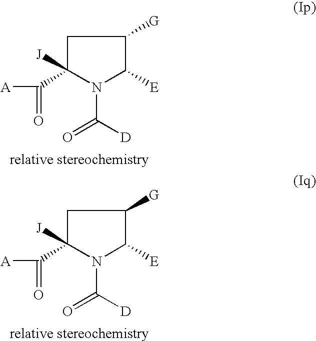
wherein A, D, E, G and J are as defined above for Formula (Ia). In a further aspect, the absolute stereochemistry of chiral compounds of Formula (Ia) is represented by Formulae (Ipp) or (Iqq): 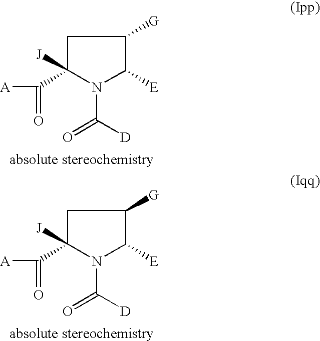
wherein A, D, E, G and J are as defined above for Formula (Ia).

The following substituent groups are preferred, where applicable, in respect of each of Formulae Ia, Ip, Ipp Iq and Iqq:

In one aspect, A is hydroxy (that is, not esterified).

In one aspect, J represents 1,3-thiazol-4-ylmethyl or 1H-pyrazol-1-ylmethyl. In a further aspect, J represents 1H-pyrazol-1-ylmethyl. In another aspect, J represents 1H-pyrazol-1-ylmethyl and E represents 1,3-thiazol-2-yl.

In one aspect, the compounds of Formula (Ia) are represented by compounds of Formula (Ipp).

It is to be understood that the present invention covers all combinations of aspects, suitable, convenient and preferred groups described herein.

The chemical entities of the present invention exhibit an improved genotype-1a/1b profile against HCV polymerase, and therefore have the potential to achieve efficacy in man over a broad patient population.

The term ‘genotype-1a/1b profile’ means potency as an inhibitor of HCV polymerase enzyme in wildtype HCV of the 1a genotype and of the 1b genotype. High potency in both genotypes is considered to be advantageous.

There is provided as a further aspect of the present invention at least one chemical entity chosen from compounds of Formula (Ia) and physiologically acceptable salts, solvates or esters thereof for use in human or veterinary medical therapy, particularly in the treatment or prophylaxis of viral infection, particularly HCV infection.

It will be appreciated that reference herein to therapy and/or treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of at least one chemical entity chosen from compounds of Formula (Ia) and physiologically acceptable salts, solvates or esters thereof in the manufacture of a medicament for the treatment and/or prophylaxis of viral infection, particularly HCV infection.

In a further or alternative aspect there is provided a method for the treatment of a human or animal subject with viral infection, particularly HCV infection, which method comprises administering to said human or animal subject an effective amount of at least one chemical entity chosen from compounds of Formula (Ia) and physiologically acceptable salts, solvates or esters thereof.

It will be appreciated that the compounds of the present invention may contain one or more asymmetric carbon atoms and may exist in racemic, diastereoisomeric, and optically active forms. All of these racemic compounds, enantiomers and diastereoisomers are contemplated to be within the scope of the present invention.

In one aspect, chemical entities useful in the present invention may be chosen from compounds of Formula (Ia) selected from the group consisting of:

rel-(2R,4S,5R)-1-(3-Methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid;

rel-(2R,4S,5R)-1-(3-Methoxy-4-tert-butylbenzoyl)-4-(methoxymethyl)-5-(1,3-thiazol-2-yl)-2-(1,3-thiazol-4-ylmethyl)pyrrolidine-2-carboxylic acid;

rel-(2R,4S,5R)-1-(3-Methoxy-4-tert-butylbenzoyl)-4-(methoxymethyl)-5-(1,3-thiazol-2-yl)-2-(1,2-thiazol-3-ylmethyl)pyrrolidine-2-carboxylic acid;

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid;

rel-(2R,4S,5R)-1-(3-Methoxy-4-tert-butylbenzoyl)-4-(methoxymethyl)-5-(5-methyl-1,3-thiazol-2-yl)-2-(1,3-thiazol-2-ylmethyl)pyrrolidine-2-carboxylic acid;

rel-(2R,4S,5R)-1-(3-Methoxy-4-tert-butylbenzoyl)-4-(methoxymethyl)-5-(5-methyl-1,3-thiazol-2-yl)-2-(1,2-thiazol-3-ylmethyl)pyrrolidine-2-carboxylic acid; and

rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid;

and salts, solvates and esters, and individual enantiomers thereof.

In a further aspect, chemical entities useful in the present invention may be chosen from compounds of Formula (Ia) selected from the group consisting of:

rel-(2R,4S,5R)-1-(3-Methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid;

rel-(2R,4S,5R)-1-(3-Methoxy-4-tert-butylbenzoyl)-4-(methoxymethyl)-5-(1,3-thiazol-2-yl)-2-(1,3-thiazol-4-ylmethyl)pyrrolidine-2-carboxylic acid;

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid;

rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid;

and salts, solvates and esters, and individual enantiomers thereof.

In a yet further aspect, chemical entities useful in the present invention may be chosen from compounds of Formula (Ia) selected from the group consisting of:

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid;

rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid;

and salts, solvates and esters, and individual enantiomers thereof.

In another aspect, chemical entities useful in the present invention may be chosen from compounds of Formula (Ia) selected from the group consisting of:

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid;

and salts, solvates and esters, and individual enantiomers thereof.

Also included in the present invention are pharmaceutically acceptable salt complexes. The present invention also covers the physiologically acceptable salts of the compounds of formula (Ia). Suitable physiologically acceptable salts of the compounds of formula (Ia) include acid salts, for example sodium, potassium, calcium, magnesium and tetraalkylammonium and the like, or mono- or di- basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (Ia), for example hydrates.

The present invention also relates to pharmaceutically acceptable esters of the compounds of Formula (Ia), for example carboxylic acid esters —COOR, in which R is selected from straight or branched chain alkyl, for example n-propyl, n-butyl, alkoxyalkyl (e.g. methoxymethyl), alkoxycarbonylalkyl (e.g. methoxycarbonylmethyl), acyloxyalkyl (e.g. pivaloyloxymethyl), aralkyl (e.g. benzyl), aryloxyalkyl (e.g. phenoxymethyl), aryl (e.g. phenyl optionally substituted by halogen, C₁₋₄alkyl or C₁₋₄alkoxy or amino). Unless otherwise specified, any alkyl moiety present in such esters preferably contains 1 to 18 carbon atoms, particularly 1 to 4 carbon atoms. Any aryl moiety present in such esters preferably comprises a phenyl group.

In one aspect, the compound of Formula (Ia) is in the form of a parent compound, a salt or a solvate.

As used herein, the term “pharmaceutically acceptable” used in relation to an ingredient (active ingredient such as an active ingredient, a salt thereof or an excipient) which may be included in a pharmaceutical formulation for administration to a patient, refers to that ingredient being acceptable in the sense of being compatible with any other ingredients present in the pharmaceutical formulation and not being deleterious to the recipient thereof.

It will further be appreciated that certain compounds of the present invention may exist in different tautomeric forms. All tautomers are contemplated to be within the scope of the present invention.

Compounds of Formula (Ia) in which A is hydroxy may be prepared from a compound of Formula (II) 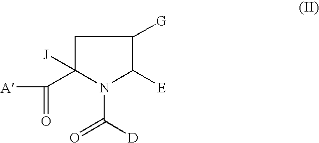
in which A′ is a protected hydroxy group, for example an alkoxy, benzyloxy or silyloxy, for example tri-(C₁₋₄alkyl)-silyloxy group, and D, E, G and J are as defined above for Formula (Ia), by deprotection. Suitable protecting groups can be found, but are not restricted to, those found in T W Greene and P G M Wuts ‘Protective Groups in Organic Synthesis’, 3^{rd} Ed (1999), J Wiley and Sons.

For example when A′ is tert-butoxy, and D, E, G and J are as defined above for Formula (la), by treatment with an appropriate acid, for example trifluoroacetic acid or aqueous concentrated hydrochloric acid solution. Optionally, the reaction is carried out in a solvent, for example dichloromethane or dimethoxyethane. In one aspect, the temperature is in the range 0 to 60° C., in a further aspect 20 to 30° C.

For example when A′ is benzyloxy, and D, E, G and J are as defined above for Formula (Ia), by hydrogenolysis in the presence of a suitable catalyst for example palladium-on-carbon. Suitably, the reaction is carried out in a solvent, for example ethanol. Preferably, the temperature is in the range 0 to 50° C.

For example when A′ is allyloxy, and D, E, G and J are as defined above for Formula (Ia), by treatment with a suitable catalyst for example tetrakis(triphenylphosphine)palladium(0) and a suitable proton source, for example phenylsilane. The reaction is carried out in a suitable solvent, for example dichloromethane.

For example when A′ is tri(methyl)silyloxy, and D, E, G and J are as defined above for Formula (Ia), by treatment with a suitable fluoride source for example tetrabutylammonium fluoride. The reaction is carried out in a suitable solvent, for example tetrahydrofuran.

Compounds of Formula (Ia) or (II) may be prepared by reaction of a compound of Formula (III) 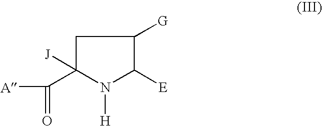
in which A″ is hydroxy or an alkoxy, benzyloxy or tri-(C₁₋₄alkyl)-silyloxy group, and E, G, and J are as defined above for Formula (Ia); with a suitable acylating agent, for example D-C(O)-hal, wherein hal is a halo atom, preferably chloro or bromo, and D is as defined above for Formula (Ia). Preferably the reaction is carried out in a suitable solvent, for example dichloromethane, in the presence of a suitable base, for example triethylamine. Suitably, the temperature is in the range 0 to 50° C., more suitably 20 to 30° C. Optionally, the reaction may be carried out at the reflux temperature of the solvent.

Compounds of Formula (Ia) or (II) may also be prepared by methylation of a compound of formula (IV) 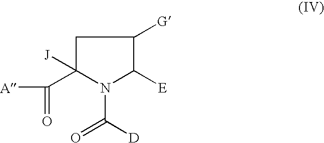
in which A″ is as defined above for Formula (III), and G′ represents hydroxymethyl using a suitable base for example sodium hydride or sodium tert-butoxide and a suitable methylating agent such as methyl iodide. In one aspect, the reaction is carried out in a suitable solvent or mixture thereof, for example dimethylformamide, methyl-tert-butyl ether, dimethoxyethane and/or acetonitrile. In one aspect, the reaction is carried out at a temperature in the range −30 to 50° C., suitably 20 to 30° C. or −25° C. In a further aspect, the reaction is carried out using a mixture of methyl-tert-butyl ether and dimethoxyethane as solvent at −25° C.

Compounds of Formula (IV) may be prepared by appropriate manipulation of a compound of Formula (V) 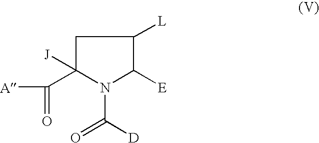
in which A″ is as defined above for Formula (III), and D, E and J are as defined above for Formula (Ia), and L represents CHO or CO₂Y wherein Y represents hydrogen or alkyl. For example, by reduction of a compound of Formula (V) in which L represents CHO or CO₂Y wherein Y represents hydrogen or alkyl, using a suitable reducing agent, for example lithium borohydride, lithium triethylborohydride, sodium borohydride, sodium triacetoxyborohydride, borane/dimethyl sulfide complex or lithium aluminium hydride, or suitable combinations thereof, in a suitable solvent or mixture thereof for example tetrahydrofuran and/or methanol. In one aspect, the reaction may be carried out at a temperature in the range −78 to 40° C. In a further aspect, the reaction may be carried out at a temperature in the range 30 to 40° C. In a further aspect, the reaction is carried out using a mixture of sodium borohydride and sodium triacetoxyborohydride in a tetrahydrofuran and methanol solvent mixture.

A compound of Formula (V) in which L represents CO₂Y wherein Y represents hydrogen may be prepared from a compound of Formula (V) in which L represents CO₂Y wherein Y represents alkyl. For example, a compound of Formula (V) in which L represents CO₂Me may be converted into a compound of Formula (V) in which L represents CO₂H by hydrolysis, for example base catalysed hydrolysis using a suitable base such as sodium methoxide in a suitable solvent such as methanol.

A compound of Formula (V) in which L represents CHO or CO₂Y wherein Y represents hydrogen or alkyl may be prepared from a compound of Formula (VI) 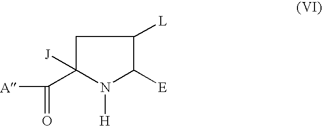
in which L represents CHO or CO₂Y wherein Y represents hydrogen or alkyl, and A″, E, and J are as defined above for Formula (III); with a suitable acylating agent, for example D-C(O)-hal, wherein hal is a halo atom, preferably chloro or bromo, and D is as defined above for Formula (Ia). Preferably the reaction is carried out in a suitable solvent, for example dichloromethane, methyl-tert-butyl ether and/or acetonitrile, in the presence of a suitable base, for example triethylamine or pyridine. In one aspect, the reaction is carried out at a temperature in the range 0 to 50° C., suitably 20 to 30° C. Optionally the reaction may be carried out under reflux. In one aspect, where pyridine is used as base, all traces of pyridine are suitably removed, for example by washing with aqueous acid, for hydrochloric acid, and/or additionally with water, before proceeding to the next synthetic step.

A compound of Formula (V) in which A″ is hydroxy, may be converted to a compound of Formula (V) in which A″ is an alkoxy, benzyloxy or silyloxy group by standard hydroxy protecting techniques. Similarly, a compound of Formula (V) in which A″ is an alkoxy, benzyloxy or silyloxy group, may be converted to a compound of Formula (V) in which A″ is hydroxy by standard deprotecting techniques. Suitable protecting groups can be found, but are not restricted to, those found in T W Greene and P G M Wuts ‘Protective Groups in Organic Synthesis’, 3^{rd} Ed (1999), J Wiley and Sons.

A compound of Formula (VI) may be prepared by reaction of a compound of Formula (VII) 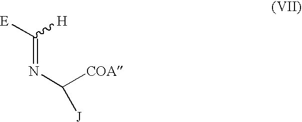
in which E and J are as defined above for Formula (Ia) and A″ is as defined above for Formula (III) with a compound of Formula (VIII) 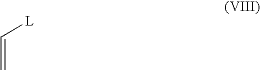
wherein L represents CHO or CO₂Y wherein Y represents hydrogen or alkyl. In one aspect, the reaction is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU), or tetramethyl guanidine. Alternatively, the reaction is carried out in a suitable solvent, for example THF or acetonitrile, in the presence of an acid, such as acetic acid, or the reaction may be carried out by heating compounds of Formula (VII) and Formula (VIII) in a suitable solvent, for example toluene, xylene or acetonitrile in the absence of a catalyst.

A compound of Formula (VI) may also be prepared in a one pot synthesis by reaction of a compound of Formula (X) with a compound of Formula (VIII) and a compound of Formula E-CHO. Preferably, the reaction is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Preferably the reaction is carried out at a temperature in the range 0 to 50° C., suitably 20 to 30° C. Optionally a drying agent is used in the process, for example molecular sieves.

A compound of Formula (III) may be prepared by appropriate manipulation of a compound of Formula (IX) 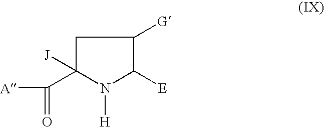
in which G′ represents hydroxymethyl, and A″, E, and J are as defined above for Formula (III), by first protecting the N-atom of the pyrrolidine ring with a suitable N-protecting group, for example benzyloxycarbonyl (CBZ) or t-butoxycarbonyl. In a similar manner to that described above in relation to conversion of compounds of Formula (IV) into compounds of Formula (II), an N-protected compound of Formula (IX) may be converted into a compound of Formula (III), in which G represents methoxymethyl and the N-atom is protected, by methylation. Deprotection of the N-atom by standard procedures results in the compound of Formula (III).

Compounds of Formula (IX) may be prepared by reduction of an optionally N-protected compound of Formula (VI) in which L represents CO₂Y and Y represents alkyl, using a suitable reducing agent, for example lithium borohydride or sodium borohydride, in a suitable solvent for example tetrahydrofuran. Deprotection of the N-atom by standard procedures results in the compound of Formula (IX). For example, when the N-protecting group is CBZ, deprotection may be achieved by catalytic hydrogenolysis. For example, when the N-protecting group is t-butoxycarbonyl, deprotection may be achieved by treatment with a suitable acid, for example trifluoroacetic acid.

Compounds of Formula (VII) may be prepared by reaction of a compound of Formula (X) 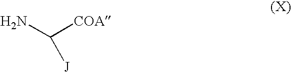
in which J is as defined above for Formula (Ia) and A″ is as defined above for Formula (III) with a compound of Formula E-CHO in which E is as defined above for Formula (Ia). In one aspect, where Formula (X) is provided as an acid addition salt, for example the hydrochloride, the reaction may be carried out in the presence of a suitable base, for example triethylamine. In a further aspect, the reaction may be carried out in the presence of a suitable drying agent, for example magnesium sulphate. The reaction may be carried out in a suitable solvent, for example dichloromethane or toluene.

Compounds of Formula (X) in which J is 1H-pyrazol-1-ylmethyl, and A″ is hydroxy, may be prepared by treatment of a compound of (XI) 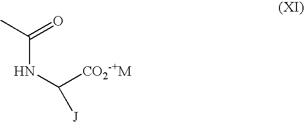
in which J is 1H-pyrazol-1-ylmethyl, and M is a metal cation, for example potassium, with a suitable acid, for example 10% aqueous hydrochloric acid, in the presence of Amberlyst 120 (H⁺).

Compounds of Formula (X) in which J is 1,3-thiazol-2-ylmethyl or 1H-pyrazol-1-ylmethyl, and A″ is an alkoxy, benzyloxy or tri-(C₁₋₄alkyl)-silyloxy group, may be prepared by treatment of a compound of Formula (X) in which J is 1,3-thiazol-2-ylmethyl or 1H-pyrazol-1-ylmethyl, and A″ is hydroxy, by conventional esterification or protecting group procedures. For example, a compound of Formula (X) in which J is 1,3-thiazol-2-ylmethyl or 1H-pyrazol-1-ylmethyl, and A″ is tert-butoxy may be prepared by treatment of a compound of Formula (X) in which J is 1,3-thiazol-2-ylmethyl or 1H-pyrazol-1-ylmethyl, and A″ is hydroxy, with an appropriate tert-butyl transfer agent, such as tert-butylacetate in the presence of a suitable acid catalyst, such as 70% aqueous perchloric acid. In one aspect, the thus-formed free base compound of Formula (X) in which A″ is an alkoxy, benzyloxy or tri-(C₁₋₄alkyl)-silyloxy group, may be converted to a suitable salt, for example the hydrochloride salt, by treatment with a suitable acid, for example hydrochloric acid in dioxane.

Compounds of Formula (XI) in which J is 1H-pyrazol-1-ylmethyl, may be prepared by reaction of a compound of Formula (XII) 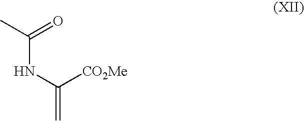
with 1H-pyrazole, in the presence of a suitable base, for example potassium carbonate when M is potassium, and in the presence of a suitable solvent, such as aqueous acetonitrile. Preferably the reaction is carried out at a temperature in the range 50-70° C., suitably 60° C.

Compounds of Formula (X) in which J is 1,3-thiazol-2-ylmethyl, 1,3-thiazol-4-ylmethyl or 1,2-thiazol-3-ylmethyl and A″ is an alkoxy, benzyloxy or tri-(C₁₋₄alkyl)-silyloxy group, may be prepared by treatment of a compound of Formula (XIII) 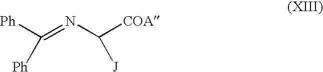
in which J is 1,3-thiazol-2-ylmethyl, 1,3-thiazol-4-ylmethyl or 1,2-thiazol-3-ylmethyl and A″ is an alkoxy, benzyloxy or tri-(C₁₋₄alkyl)-silyloxy group with an acid, for example 15% aqueous citric acid. Preferably, the reaction is carried out in a suitable solvent, for example THF and/or water.

Compounds of Formula (XIII) may be prepared by reaction of a compound of Formula (XIV) 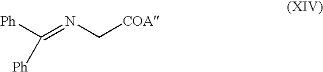
in which A″ is an alkoxy, benzyloxy or tri-(C₁₋₄alkyl)-silyloxy group with a compound of Formula J-hal in which J is 1,3-thiazol-2-ylmethyl, 1,3-thiazol-4-ylmethyl or 1,2-thiazol-3-ylmethyl, and hal is a halo atom, preferably chloro or bromo. Preferably, the reaction is carried out in the presence of a suitable base such as potassium t-butoxide. Preferably, the reaction is carried out in a suitable solvent, for example THF. The reaction may be carried out in the presence of a suitable catalyst, for example lithium iodide. Preferably the reaction is carried out at a temperature in the range −10° C. to room temperature, suitably at 0° C.

The compound of Formula D-C(O)-hal in which D is 3-methoxy-4-tert-butylphenyl may be prepared by reaction of a compound of Formula (XV)

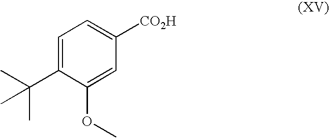

with a suitable acid halide forming reagent, for example oxalyl chloride or thionyl chloride. In one aspect, the reaction is carried out in the presence of a suitable catalyst, for example dimethylformamide or diethylformamide. Optionally, the reaction is carried out in a suitable solvent, for example dichloromethane, at a temperature in the range 0 to 50° C., for example 20 to 30° C. In an alternative aspect, the reaction is carried out using thionyl chloride under reflux.

Compounds of Formula (VIII), (XII), (XIV), (XV), J-hal and E-CHO are known in the art, commercially available, or may be prepared by standard literature procedures.

Compounds of Formula (Ia) in which A is an ester may be prepared by esterification of a compound of Formula (Ia) in which A is hydroxy by standard literature procedures for esterification.

It will be appreciated that compounds of Formula (Ia), (II), (III), (IV), (V), (VI) and/or (IX) which exist as diastereoisomers may optionally be separated by techniques well known in the art, for example by column chromatography.

It will also be appreciated that the present invention provides a method for the interconversion of C(4)-epimers of a compound of formula (V) or (VI) in which L represents CHO or CO₂Y wherein Y represents hydrogen or alkyl, and A″, E, and J are as defined above for formula (III). For example the rel-(2R, 4S, 5R)-diastereoisomer of a compound of formula (V) and/or (VI) may be converted into the rel-(2R, 4R, 5R)-diastereoisomer where appropriate, Such epimerisation of these rel-(4S, 5R)-diasteroisomers into the corresponding rel-(4R, 5R)-diastereoisomers may be accomplished by treatment of a compound of formula (V) and/or (VI) with a suitable base, in the presence of a suitable solvent. For example the conversion of the rel-(4S, 5R)-diastereoisomer of a compound of Formula (V) when L represents CO₂Me into the rel-(4R, 5R)-diastereoisomer may be accomplished by treatment of the rel-(4S, 5R)-diastereoisomer with a suitable base, such as sodium methoxide, in the presence of a suitable solvent, such as methanol.

It will be appreciated that racemic compounds of Formula (Ia), (II), (III), (IV), (V), (VI) and/or (IX) may be optionally resolved into their individual enantiomers. Such resolutions may conveniently be accomplished by standard methods known in the art. For example, a racemic compound of Formula (Ia), (II), (III), (IV), (V), (VI) and/or (IX) may be resolved by chiral preparative HPLC. Alternatively, racemic compounds of Formula (Ia), (II), (III), (IV), (V), (VI) and/or (IX) which contain an appropriate acidic or basic group, such as a carboxylic acid group or amine group may be resolved by standard diastereoisomeric salt formation with a chiral base or acid reagent respectively as appropriate. Such techniques are well established in the art. For example, a racemic compound of Formula (VI) where L is CO₂Me may be resolved by treatment with a chiral acid such as (R)-(−)-1,1′-binaphthyl-2,2′-diyl-hydrogen phosphate, in a suitable solvent or mixture thereof, for example dichloromethane, isopropanol, isopropyl acetate and/or acetonitrile. In one aspect a mixture of dichloromethane and isopropyl acetate is used as solvent. The enantiomer of Formula (VI) may then be obtained by treating the salt with a suitable base, for example triethylamine, in a suitable solvent, for example methyl tert-butyl ether. Individual enantiomers of Formula (II), (III), (IV), (V), (VI) and/or (IX) may then be progressed to an enantiomeric compound of Formula (Ia) by the chemistry described above in respect of racemic compounds.

It will also be appreciated that individual enantiomeric compounds of Formula (III), (VI) and/or (IX) may be prepared by general methods of asymmetric synthesis using, where appropriate, chiral auxiliaries or chiral catalytic reagents and additionally performing any suitable functional group interconversion step as hereinbefore described, including the addition or removal of any such chiral auxiliary. Such general methods of asymmetric synthesis are well known in the art and include, but are not restricted to, those described in “Asymmetric Synthesis,” Academic Press, 1984 and/or “Chiral Auxiliaries and Ligands in Asymmetric Synthesis”, Wiley, 1995. For example, suitable general chiral auxiliaries include chiral alcohols such as menthol or 1-phenylethanol; chiral oxazolidinones such as 4-benzyloxazolidin-2-one or 4-isopropyloxazolidin-2-one; chiral sultams such as camphor sultam; or chiral amines such as 1-phenylethylamine or 2-amino-2-phenylethanol. Suitable general chiral catalytic reagents include chiral basic amines and chiral ligands such as N-methylephedrine, 1-phenyl-2-(1-pyrrolidinyl)-1-propanol, 3-(dimethylamino)-1,7,7-trimethylbicyclo[2.2.1 ]-heptan-2-ol, 3,4-bis(diphenylphosphanyl)-1-(phenylmethyl)-pyrrolidine, chinchonine, chinchonidine, sparteine, hydroquinine or quinine, BINAP or chiral bis(oxazoline) (BOX) ligands and derivatives, optionally in the presence of a metal salt, for example MₘXₓ where M is silver, cobalt, zinc, titanium, magnesium, or manganese, and X is halide (for example chloride or bromide), acetate, trifluoroacetate, p-toluenesulfonate, trifluoromethylsulfonate, hexafluorophosphate or nitrate, and m and x are 1, 2, 3 or 4, and optionally in the presence of a base, for example triethylamine. All of these chiral auxiliaries or chiral catalytic reagents are well described in the art. General illustrative examples of the preparation of various chiral pyrrolidines by asymmetric synthesis using chiral auxiliaries or chiral catalytic reagents include, but are not limited to, those described in Angew. Chem. Int. Ed., (2002), 41: 4236; Chem. Rev., (1998), 98: 863; J. Am. Chem. Soc., (2002), 124: 13400; J. Am. Chem. Soc., (2003), 125: 10175; Org. Lett., (2003), 5, 5043; Tetrahedron, (1995), 51: 273; Tetrahedron: Asymm., (1995), 6: 2475; Tetrahedron: Asymm., (2001), 12: 1977; Tetrahedron: Asymm., (2002), 13: 2099 and Tet Lett., (1991), 41: 5817.

In a particular aspect, a chiral pyrrolidine compound of Formula (VIa) 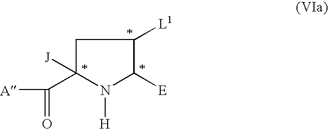
in which L¹ represents CO₂Y or CO₂Y¹ wherein Y represents hydrogen or alkyl, Y¹ represents a chiral auxiliary, and A″, E, and J are as defined above for Formula (VI), and * denotes an enantioenriched chiral centre can be prepared by reaction of a compound of Formula (VII), as hereinbefore defined, with a compound of Formula (VIIIa) 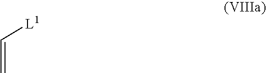
in which L¹ represents a chiral ester group CO₂Y¹ wherein Y¹ represents a chiral auxiliary and thereafter optionally carrying out any conversion of CO₂Y¹ into CO₂Y by standard methods for removal of chiral auxiliaries. Such chiral ester CO₂Y¹ may be derived from a chiral alcohol Y¹OH, for example menthol, by standard esterification techniques. Preferably, the reaction of a compound of Formula (VII) with a compound of Formula (VIIIa) is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Alternatively, the reaction is carried out in a suitable solvent, for example THF or acetonitrile, in the presence of an acid, such as acetic acid, or the reaction may be carried out by heating compounds of Formula (VII) and (VIIIa) in a suitable solvent, for example toluene, xylene or acetonitrile in the absence of a catalyst. The preparation of compounds analogous to those of Formula (VIa) and (VIIIa) is described in Tetrahedron: Asymm., (1995), 6: 2475.

In a further aspect, a chiral pyrrolidine compound of Formula (VIb) 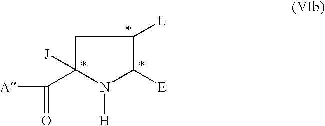
in which L represents CO₂Y wherein Y represents hydrogen or alkyl, and A″, E, and J are as defined above for Formula (VI), and * denotes an enantioenriched chiral centre can be prepared by reaction of a compound of Formula (VII) with a compound of Formula (VIII) as herein before defined, under asymmetric reaction conditions. It will be appreciated by those skilled in the art that such asymmetric reaction conditions may be afforded by, for example, the inclusion in the reaction mixture of a chiral catalytic reagent as herein before defined.

In one aspect, the reaction is carried out in the presence of a suitable chiral catalytic reagent, for example (−)-N-methylephedrine, and a suitable metal salt, for example manganese (II) bromide, in a suitable solvent, for example acetonitrile. Preferably the reaction is carried out at a temperature in the range −30° C. to room temperature, suitably at −20° C.

In an alternative aspect, the reaction is carried out in the presence of a suitable chiral catalytic reagent, for example (S)-(−)-2,2′-bis(diphenylphosphino)-1′1-binaphthyl (S-BINAP), and a suitable metal salt, for example silver acetate, in the presence of a suitable base, for example diisopropylethylamine, in a suitable solvent, for example acetonitrile optionally co-solvated with toluene. Preferably the reaction is carried out at a temperature in the range −15° C. to room temperature, suitably at −5° C.

Optionally, the major chiral diastereoisomer of a compound of Formula (VIa) or Formula (VIb) arising from such an asymmetric reaction may be further enantioenriched by conventional purification techniques well known in the art, for example by chromatography, or by fractional crystallisation. A favourable crystallisation method is the fractional crystallisation of a salt of the major chiral diastereoisomer, for example the hydrochloride salt or the (R)-(−)-1,1′-binaphthyl-2,2′-diyl-hydrogen phosphate salt. The hydrochloride salt of a compound of Formula (VIa) or Formula (VIb) may be prepared by treating a compound of Formula (VIa) or Formula (VIb) with anhydrous hydrogen chloride in a suitable solvent, for example diethyl ether. Preferably the reaction is carried out at a temperature in the range −10 to 10° C.

The (R)-(−)-1,1′-binaphthyl-2,2′-diyl-hydrogen phosphate salt of a compound of Formula (VIa) or Formula (VIb) may be prepared as herein before described for the resolution of a racemic compound of Formula (VI).

Optional removal of a chiral auxiliary from a group in which L¹ represents CO₂Y¹ to afford a group in which L¹ represents CO₂Y is readily accomplished by standard methods, for example treatment with a hydrolytic reagent such as sodium hydroxide or an alkoxide such as sodium methoxide as appropriate, in a suitable solvent such as methanol.

Optionally, a chiral compound of Formula (VIa) or Formula (VIb) may be converted into a chiral compound of Formula (IX) in which G′ represents hydroxyalkyl, and A″, E, and J are as defined above for Formula (III) by treatment with suitable reagents for accomplishing the functional group interconversion of the group L or L¹ into group G′. For example a compound of Formula (VIa) in which L¹ represents CO₂Y¹ and Y¹ is as defined above may be treated with a suitable reducing agent, for example lithium aluminium hydride, in a suitable solvent, for example tetrahydrofuran.

Optionally, a chiral compound of Formula (VIa) or Formula (VIb) may be converted into a chiral compound of Formula (IV) in which G′ represents hydroxyalkyl, by first acylating the pyrrolidine nitrogen atom as described above for the transformation of a compound of Formula (VI) into a compound of Formula (V) and then subsequently by treatment with suitable reagents for accomplishing the functional group interconversion of the group L or L¹ into group G′ as described above for the transformation of a compound of Formula (VIa) or Formula (VIb) into a chiral compound of Formula (IX).

It will be appreciated that, with suitable additional conversion steps as described above, chiral compounds of Formula (Ia), (II), (IV) and/or (V) may be prepared from chiral compounds of Formula (III), (VI) and (IX).

With appropriate manipulation and protection of any chemical functionality, synthesis of compounds of Formula (I) is accomplished by methods analogous to those above and to those described in the Experimental section. Suitable protecting groups can be found, but are not restricted to, those found in T W Greene and P G M Wuts ‘Protective Groups in Organic Synthesis’, 3^{rd} Ed (1999), J Wiley and Sons.

### EXAMPLES

It will be appreciated by those skilled in the art that when solvents are used in reactions it is desirable to use anhydrous solvents. It is further desirable to conduct reactions under an inert atmosphere, for example under nitrogen or argon, where appropriate.

Intermediate 1
2-[N-(Diphenylmethylene)amino]-3-(1,3-thiazol4-yl)propanoic acid, tert-butyl ester

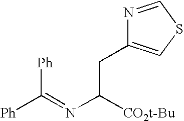

Part A

To a cooled (ice-bath) solution of 2-[N-(diphenylmethylene)amino]ethanoic acid, tert-butyl ester (J. Org. Chem., 1982, 47, 2663; 42.3 g, 143 mmol) in dry THF (450 mL) under an atmosphere of nitrogen, was added a 1M solution of potassium t-butoxide in THF (146 mL) dropwise (dropping funnel) over 25 minutes. The mixture was allowed to stir for a further 45 minutes in the ice-bath.

Part B

Independently during this time, 4-(chloromethyl)-1,3-thiazole hydrochloride (25.5 g, 150 mmol) was freshly converted to the free base as follows: The hydrochloride was mixed with dichloromethane (500 mL) and washed with a 5% w/v aqueous sodium bicarbonate solution (375 mL). The organic layer was separated, dried over sodium sulphate and carefully evaporated (rotary evaporator; 80 torr, water bath 25° C.) to give the free base.

Part C

The 4-(chloromethyl)-1,3-thiazole (formed in Part B) was dissolved in THF (100 mL) and added dropwise (dropping funnel) over 30 minutes to the reaction mixture from Part A, keeping the reaction at ice-bath temperature. Solid anhydrous lithium iodide (1 g, 7.5 mmol) was added directly to the reaction mixture 5 minutes after addition of the alkylating agent had started. The dropping funnel was rinsed with further dry THF (50 mL) which was added to the reaction. The reaction was stirred at ice-bath temperature for 45 minutes, allowed to warm to room temperature over 30 minutes and was stirred at room temperature for an additional 2.5 hours before being partitioned between a mixture of saturated brine (400 mL), water (200 mL) and ethyl acetate (800 mL). The organic layer was separated and the aqueous layer re-extracted with further ethyl acetate (2×300 mL). The combined organic layers were dried over sodium sulphate and evaporated to give the title compound (57.8 g, crude) which was used without further purification.

¹H NMR (CDCl₃): δ 8.65 (d, 1H), 7.55-7.62 (m, 2H), 7.2-7.55 (m, 6H), 7.05 (d, 1H), 6.78-6.87 (m, 2H), 4.36-4.41 (m, 1H), 3.47-3.54 (m, 1H), 3.36-3.44 (m, 1H) and 1.44 (s, 9H).

Intermediate 2
2-Amino-3-(1,3-thiazol-4-yl)propanoic acid, tert-butyl ester

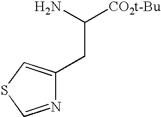

To a solution of 2-[N-(diphenylmethylene)amino]-3-(1,3-thiazol-4-yl)propanoic acid, tert-butyl ester (prepared in a similar manner to that described in Intermediate 1; 20 g) in THF (150 mL) under argon was added a solution of citric acid in water (15% w/v, 150 mL). The mixture was stirred at room temperature for 6 hours, left overnight and then the majority of the THF was removed under reduced pressure (rotary evaporator; water bath at 25° C.) and 1 M aqueous hydrochloric acid (60 mL) added. The mixture was extracted with diethyl ether (2×200 mL) and the combined ether extracts back extracted with water (50 mL). The combined aqueous layers were extracted with further diethyl ether (100 mL). All of the ether layers were discarded. The aqueous layer was then carefully adjusted to pH 9.5 with potassium carbonate, brine (100 mL) was added and the mixture extracted with diethyl ether (4×200 mL). These combined ether layers were dried over sodium sulphate. Removal of the solvent under reduced pressure gave the title compound, an oil.

¹H NMR (CDCl₃): δ 8.77 (d, 1H), 7.08 (d, 1H), 3.77-3.85 (m, 1H), 3.22-3.32 (m, 1H), 3.02-3.13 (m,1H) and 1.42 (s, 9H). Amine protons not observed.

Intermediate 3
2-[[N-(5-Methyl-1,3-thiazol-2-yl)methylene]amino]-3-(1,3-thiazol-4-yl)propanoic acid, tert-butyl ester

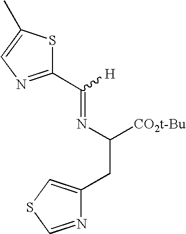

A mixture of 2-amino-3-(1,3-thiazol-4-yl)propanoic acid, tert-butyl ester (Intermediate 2; 2.90 g, 12.7 mmol), 5-methyl-1,3-thiazole-2-carboxaldehyde (1.62 g, 12.7 mmol) and magnesium sulfate (ca. 1 g) in dichloromethane (70 mL) was stirred at room temperature for 18 hours. The reaction mixture was filtered, and the filtrate was evaporated to remove solvent, to give the title compound as an oil.

¹H NMR (CDCl₃): δ 8.75 (d, 1H), 8.11 (s, 1H), 7.55(br, 1H), 7.02 (d, 1H), 4.45 (dd, 1H), 3.56 (dd,1H), 3.33 (dd,1H), 2.50 (br s, 3H) and 1.44 (s, 9H).

Intermediate 4
rel-(2R,4S,5R)-5-(5-Methyl-1,3-thiazol-2-yl)-2-(1,3-thiazol-4-ylmethyl)pyrrolidine-2,4-dicarboxylic acid, 2-tert-butyl ester, 4-methyl ester

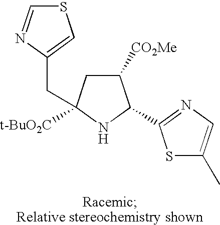

A solution of 2-[[N-(5-methyl-1,3-thiazol-2-yl)methylene]amino]-3-(1,3-thiazol-4-yl)-propanoic acid, tert-butyl ester (Intermediate 3, 4.3g, 12.75 mmol) in tetrahydrofuran (125 mL) was stirred under nitrogen. Methyl acrylate (2.15 g, 25 mmol), lithium bromide (2.20 g, 25 mmol) and triethylamine (1.73 mL, 12.5 mmol) were added successively, and the resulting mixture was stirred for 18 hours. Saturated ammonium chloride solution (100 mL) was added and the mixture was extracted with ethyl acetate (2×150 mL). Combined extracts were washed with water (100 mL) and saturated brine (100 mL), dried over magnesium sulfate and evaporated. The residue was purified by chromatography on silica gel using ethyl acetate-cyclohexane (1:1 v/v then 2:1 v/v) as eluent to give the title compound as an oil.

MS calcd for (C₁₉H₂₅N₃O₄S₂+H)⁺: 424.

MS found (electrospray): (M+H)⁺=424

Intermediate 5
rel-(2R,4S,5R)-1-(3-Methoxy-4-tert-butylbenzoyl)-5-(5-methyl-1,3-thiazol-2-yl)-2-(1,3-thiazol-4-ylmethyl)pyrrolidine-2,4-dicarboxylic acid, 2-tert-butyl ester, 4-methyl ester

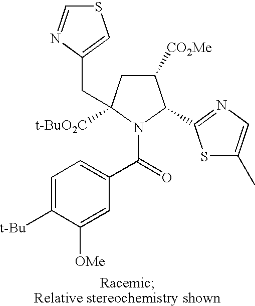

A mixture of rel-(2R,4S,5R)-5-(5-methyl-1,3-thiazol-2-yl)-2-(1,3-thiazol-4-ylmethyl)-pyrrolidine-2,4-dicarboxylic acid, 2-tert-butyl ester, 4-methyl ester (Intermediate 4, 3.90 g, 9.2 mmol) in dichloromethane (50 mL), 3-methoxy-4-tert-butylbenzoyl chloride¹ (3.28 g, 15.6 mmol) and triethylamine (3.52 mL) was stirred at room temperature under nitrogen for 3 days. The mixture was washed with water (100 mL) and brine (50 mL), dried by passage through a hydrophobic frit and evaporated. The residue was purified by chromatography on silica gel using cyclohexane-ethyl acetate (3:1 v/v) as eluent to give the title compound as a foam.

MS calcd for (C₃₁H₃₉N₃O₆S₂+H)⁺: 614

MS found (electrospray): (M+H)⁺=614.

Ref. (1): Synthesised from 3-methoxy-4-tert-butylbenzoic acid (J. Org. Chem., 26, 1961, 1732-1737).

Intermediate 6
rel-(2R,4S,5R)-4-(Hydroxymethyl)-1-(3-methoxy-4-tert-butylbenzoyl)-5-(5-methyl-1,3-thiazol-2-yl)-2-(1,3-thiazol-4-ylmethyl)pyrrolidine-2-carboxylic acid, tert-butyl ester

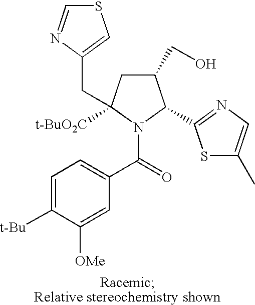

A solution of rel-(2R,4S,5R)-1-(3-methoxy-4-tert-butylbenzoyl)-5-(5-methyl-1,3-thiazol-2-yl)-2-(1,3-thiazol-4-ylmethyl)pyrrolidine-2,4-dicarboxylic acid, 2-tert-butyl ester, 4-methyl ester (Intermediate 5, 4.80 g, 7.82 mmol) in tetrahydrofuran (200 mL) was stirred under nitrogen and cooled to −78° C. A 1.0 M solution of lithium aluminium hydride in diethyl ether (8.1 mL) was added dropwise. When addition was complete the mixture was warmed to −45° C. and held at that temperature for 3.5 hours. The mixture was quenched with 1M aqueous potassium carbonate solution (100 mL) and extracted with ethyl acetate (2×200 mL). Extracts were washed with water (100 mL) and brine (100 mL), dried over magnesium sulfate and evaporated. The residue was purified by chromatography on silica gel using cyclohexane-ethyl acetate (initially 2:1 v/v then 1:2 v/v) as eluent to give the title compound as a foam.

MS calcd for (C₃₀H₃₉N₃O₅S₂+H)⁺: 586

MS found (electrospray): (M+H)⁺=586

Intermediate 7
rel-(2R,4S,5R)-1-(3-Methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid, tert-butyl ester

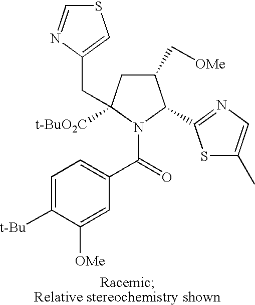

A solution of rel-(2R,4S,5R)-4-(hydroxymethyl)-1-(3-methoxy-4-tert-butylbenzoyl)-5-(5-methyl-1,3-thiazol-2-yl)-2-(1,3-thiazol-4-ylmethyl)pyrrolidine-2-carboxylic acid, tert-butyl ester (Intermediate 6, 0.585 g, 1 mmol) in N,N-dimethylformamide (10 mL) was stirred under nitrogen and cooled to −10° C. A 60% dispersion of sodium hydride in mineral oil (69 mg, 1.725 mmol) was added and the resulting mixture was stirred below 0° C. for 30 min. Iodomethane (0.425 mL) was added and the mixture was stirred for a further 4 hours, maintaining the temperature of the reaction mixture between 0° C. and 20° C. The mixture was quenched with methanol (10 mL) and evaporated. The residue was partitioned between water (50 mL) and ethyl acetate (100 mL). The organic layer was collected, washed with water and brine, dried over magnesium sulfate and evaporated. The residue was purified by chromatography on silica gel using cyclohexane-ethyl acetate (initially 2:1 v/v then 1:1 v/v) as eluent to give the title compound as a foam.

MS calcd for (C₃₁H₄₁N₃O₅S₂+H)⁺: 600

MS found (electrospray): (M+H)⁺=600

Intermediate 8
2-[N-(1,3-Thiazol-2-ylmethylene)amino]-3-(1,3-thiazol-4-yl)propanoic acid, tert-butyl ester

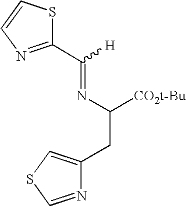

The title compound was prepared in a similar manner to Intermediate 3, using 1,3-thiazole-2-carboxaldehyde in place of 5-methyl-1,3-thiazole-2-carboxaldehyde.

¹NMR (CDCl₃): δ 8.75 (d, 1H), 8.23 (s, 1H), 7.90 (d, 1H), 7.43 (dd, 1H), 7.03 (d, 1H), 4.50 (dd, 1H), 3.58 (dd, 1H), 3.34 (dd, 1H) and 1.45 (s, 9H).

Intermediate 9
rel-(2R,4S,5R)-5-(1,3-Thiazol-2-yl)-2-(1,3-thiazol-4-ylmethyl)pyrrolidine-2,4-dicarboxylic acid, 2-tert-butyl ester, 4-methyl ester

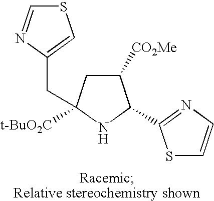

The title compound was prepared in a similar manner to Intermediate 4, using Intermediate 8 in place of Intermediate 3. The mixture was cooled to 0° C. prior to addition of methyl acrylate, and allowed to warm to RT before quenching.

MS calcd for (C₁₈H₂₃N₃O₄S₂+H)⁺: 410

MS found (electrospray): (M+H)⁺=410

Intermediate 10
rel-(2R,4S,5R)-1-(3-Methoxy-4-tert-butylbenzoyl)-5-(1,3-thiazol-2-yl)-2-(1,3-thiazol-4-ylmethyl)pyrrolidine-2,4-dicarboxylic acid, 2-tert-butyl ester, 4-methyl ester

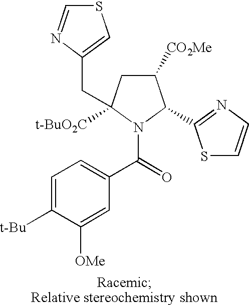

The title compound was prepared in a similar manner to Intermediate 5, using Intermediate 9 in place of Intermediate 4. Cyclohexane - ethyl acetate (gradient elution from 50:1 to 1:1) was used as the eluent for chromatography.

MS calcd for (C₃₀H₃₇N₃O₆S₂+H)⁺: 600

MS found (electrospray): (M+H)⁺=600

Intermediate 11
rel-(2R,4S,5R)-4-(Hydroxymethyl)-1-(3-methoxy-4-tert-butylbenzoyl)-5-(1,3-thiazol-2-yl)-2-(1,3-thiazol-4-ylmethyl)pyrrolidine-2-carboxylic acid, tert-butyl ester

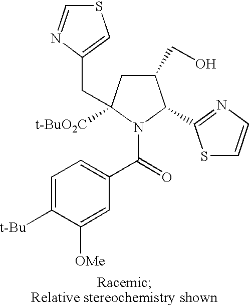

The title compound was prepared in a similar manner to Intermediate 6, using Intermediate 10 in place of Intermediate 5. Cyclohexane—ethyl acetate (gradient elution from 50:1 to 2:3) was used as the eluent for chromatography.

MS calcd for (C₂₉H₃₇N₃O₅S₂+H)⁺: 572

MS found (electrospray): (M+H)⁺=572

Intermediate 12
rel-(2R,4S,5R)-1-(3-Methoxy-4-tert-butylbenzoyl)-4-(methoxymethyl)-5-(1,3-thiazol-2-yl)-2-(1,3-thiazol-4-ylmethyl)pyrrolidine-2-carboxylic acid, tert-butyl ester

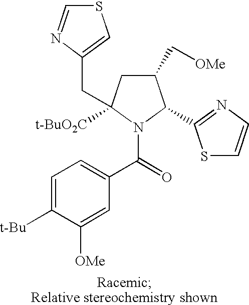

The title compound was prepared in a similar manner to Intermediate 7, using Intermediate 11 in place of Intermediate 6. Cyclohexane—ethyl acetate (gradient elution from 50:1 to 1:1) was used as the eluent for chromatography.

MS calcd for (C₃₀H₃₉N₃O₅S₂+H)⁺: 586

MS found (electrospray): (M+H)⁺=586

Intermediate 13
2-[N-(Diphenylmethylene)amino]-3-(1,2-thiazol-3-yl)propanoic acid, tert-butyl ester

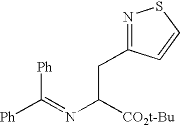

The title compound was prepared in a similar manner to Intermediate 1, using 3-(bromomethyl)-1,2-thiazole in place of 4-(chloromethyl)-1,3-thiazole.

¹NMR (CDCl₃): δ 8.51 (d, 1H), 7.61-7.18 (m, 8H), 7.08 (dd, 1H), 6.83 (m, 2H), 4.38 (dd, 1H), 3.48 (d, 2H) and 1.43 (s, 9H).

Intermediate 14
2-Amino-3-(1,2-thiazol-3-yl)propanoic acid, tert-butyl ester

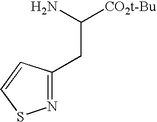

The title compound was prepared in a similar manner to Intermediate 2, using Intermediate 13 in place of Intermediate 1.

¹HMR (CDCl₃): δ 8.60 (d, 1H), 7.13 (d, 1H), 3.84 (dd, 1H), 3.29 (dd, 1H), 3.15 (dd, 1H) and 1.43 (s, 9H). Amine protons not seen.

Intermediate 15
2-[N-(1,3-Thiazol-2-ylmethylene)amino]-3-(1,2-thiazol-3-yl)propanoic acid, tert-butyl ester

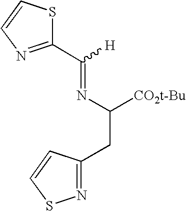

The title compound was prepared in a similar manner to Intermediate 3, using Intermediate 14 in place of Intermediate 2, and 1,3-thiazole-2-carboxaldehyde in place of 5-methyl-1,3-thiazole-2-carboxaldehyde . The reaction was heated under reflux for 1.5 hours.

¹H NMR (CDCl₃): δ 8.54 (d, 1H), 8.34 (s, 1H), 7.92 (d, 1H), 7.44 (dd, 1H), 7.10 (d, 1H), 4.54 (dd, 1H), 3.60 (dd, 1H), 3.42 (dd, 1H) and 1.43 (s, 9H).

Intermediate 16
rel-(2R,4S,5R)-2-(1,2-Thiazol-3-ylmethyl)-5-(1,3-Thiazol-2-yl)pyrrolidine-2,4-dicarboxylic acid, 2-tert-butyl ester, 4-methyl ester

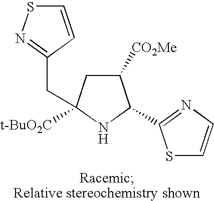

The title compound was prepared in a similar manner to Intermediate 4, using Intermediate 15 in place of Intermediate 3. Cyclohexane - ethyl acetate (3:2 v/v) was used as the eluent for chromatography, followed by evaporation of the solvent to provide the title compound as a foam.

¹H NMR (CDCl₃): δ 8.58 (d, 1H), 7.66 (d, 1H), 7.24 (d, 1H), 7.22 (d, 1H), 4.85 (d, 1H), 3.46 (s, 3H), 3.34(d, 1H), 3.28-3.22 (m, 1H), 3.20 (d, 1H), 2.85 (dd, 1H), 2.31 (dd, 1H) and 1.43 (s, 9H). Amine proton not seen.

Intermediate 17
rel-(2R,4S,5R)-1-(3-Methoxy-4-tert-butylbenzoyl)-2-(1,2-thiazol-3-ylmethyl)-5-(1,3-thiazol-2-yl)pyrrolidine-2,4-dicarboxylic acid, 2-tert-butyl ester, 4-methyl ester

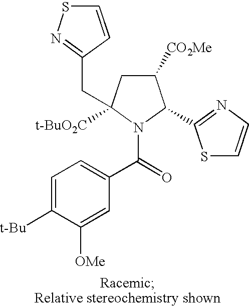

The title compound was prepared in a similar manner to Intermediate 5, using Intermediate 16 in place of Intermediate 4. After the brine wash, saturated aqueous sodium bicarbonate was added with stirring for 25 mins. Cyclohexane - ethyl acetate (4:1 v/v) was used as the eluent for chromatography.

MS calcd for (C₃₀H₃₇N₃O₆S₂+H)⁺: 600

MS found (electrospray): (M+H)⁺=600

Intermediate 18
rel-(2R,4S,5R)-4-(Hydroxymethyl)-1-(3-methoxy-4-tert-butylbenzoyl)-2-(1,2-thiazol-3-ylmethyl)-5-(1,3-thiazol-2-yl)pyrrolidine-2-carboxylic acid, tert-butyl ester

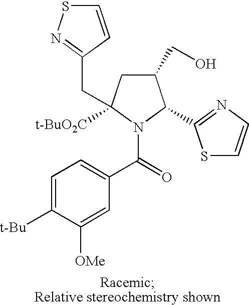

The title compound was prepared in a similar manner to Intermediate 6, using Intermediate 17 in place of Intermediate 5 and ammonium chloride in place of aqueous potassium carbonate.

MS calcd for (C₂₉H₃₇N₃O₅S₂+H)⁺: 572

MS found (electrospray): (M+H)⁺=572

Intermediate 19
rel-(2R,4S,5R)-1-(3-Methoxy-4-tert-butylbenzoyl)-4-(methoxymethyl)-2-(1,2-thiazol-3-ylmethyl)-5-(1,3-thiazol-2-yl)pyrrolidine-2-carboxylic acid, tert-butyl ester

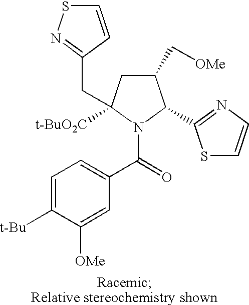

The title compound was prepared in a similar manner to Intermediate 7, using Intermediate 18 in place of Intermediate 6.

¹H NMR (CDCl₃): δ 8.66 (d, 1H), 7.42 (d, 1H), 7.32 (d, 1H), 7.15 (d, 1H), 7.10 (d, 1H), 6.71 (d, 1H), 6.52 (s, 1H), 5.17 (d, 1H), 4.18 (d, 1H), 3.61 (d, 1H), 3.63 (s, 3H), 2.94 (s, 3H), 2.76 (dd, 1H), 2.66 (dd, 1H), 2.45 (dd, 1H), 2.34 (t, 1H), 2.02-1.91 (m, 1H), 1.64 (s, 9H) and 1.29 (s, 9H).

Intermediate 20
2-Amino-3-(1H-pyrazol-1-yl)propanoic acid, tert-butyl ester

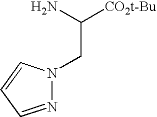

To a stirred suspension of 2-amino-3-(1H-pyrazol-1-yl)propanoic acid (10.2 g, 65.9 mmol) in tert-butyl acetate (400 mL) was added a solution of 70% aqueous perchloric acid (15.7 mL). The mixture was allowed to stir at room temperature for 30 minutes and was then allowed to stand for 20 hours. The reaction mixture was diluted with ethyl acetate and then neutralised using a combination of saturated aqueous sodium bicarbonate and solid sodium bicarbonate. The aqueous phase was separated off and extracted with ethyl acetate. The organic phases were combined, dried over sodium sulfate and evaporated to give the title compound as an oil.

MS calcd for (C₁₀H₁₇N₃O₂+H)⁺: 212

MS found (electrospray): (M+H)⁺=212.

Intermediate 21
2-[N-(1,3-Thiazol-2-ylmethylene)amino]-3-(1H-pyrazol-1-yl)propanoic acid, tert-butyl ester

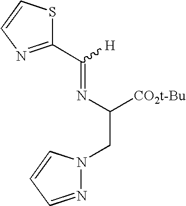

The title compound was prepared in a similar manner to Intermediate 3, using 1,3-thiazole-2-carboxaldehyde in place of 5-methyl-1,3-thiazole-2-carboxaldehyde and using Intermediate 20 in place of Intermediate 2.

¹H NMR (CDCl₃): δ 8.09 (s 1H), 7.90 (d, 1H), 7.51 (d, 1H), 7.42 (d, 1H), 7.33 (d, 1H), 6.12 (t, 1H), 4.78 (dd, 1H), 4.50 (dd, 2H), 1.46 (s, 9H).

Intermediate 22
rel-(2R,4S,5R)-2-(1H-Pyrazol-1-ylmethyl)-5-(1,3-thiazol-2-yl)pyrrolidine-2,4-dicarboxylic acid, 2-tert-butyl ester, 4-methyl ester

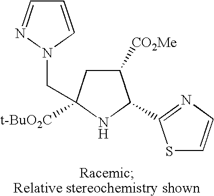

The title compound was prepared in a similar manner to Intermediate 4, using Intermediate 21 in place of Intermediate 3. Ethyl acetate—cyclohexane (1:2 then 1:1 v/v) was used as the eluent for chromatography.

MS calcd for (C₁₈H₂₄N₄O₄S+H)⁺: 393

MS found (electrospray): (M+H)⁺=393

Intermediate 23
rel-(2R,4S,5R)-1-(3-Methoxy-4-tert-butylbenzoyl)-2-(1H-pyrazol-1-ylmethyl)-5-(1,3-thiazol-2-yl)pyrrolidine-2,4-dicarboxylic acid, 2-tert-butyl ester, 4-methyl ester

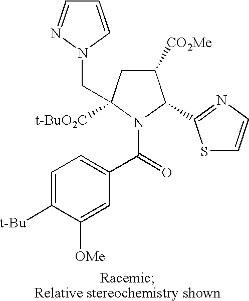

The title compound was prepared in a similar manner to Intermediate 5, using Intermediate 22 in place of Intermediate 4.

MS calcd for (C₃₀H₃₈N₄O₆S+H)⁺: 583

MS found (electrospray): (M+H)⁺=583

Intermediate 24
rel-(2R,4S,5R)-4-Hydroxymethyl-1-(3-methoxy-4-tert-butylbenzoyl)-2-(1H-pyrazol-1-ylmethyl)-5-(1,3-thiazol-2-yl)pyrrolidine-2,4-dicarboxylic acid, 2-tert-butyl ester

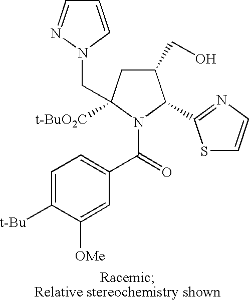

The title compound was prepared in a similar manner to Intermediate 6, using Intermediate 23 in place of Intermediate 5. Cyclohexane - ethyl acetate (gradient elution from 50:1 to 1:2 v/v) was used as the eluent for chromatography.

MS calcd for (C₂₉H₃₈N₄O₅S+H)⁺: 555

MS found (electrospray): (M+H)⁺=555

Intermediate 25
rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2,4-dicarboxylic acid, 2-tert-butyl ester

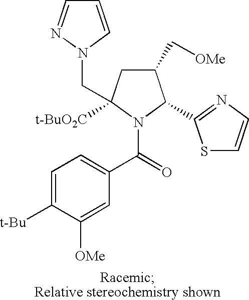

The title compound was prepared in a similar manner to Intermediate 7, using Intermediate 24 in place of Intermediate 5. Cyclohexane - ethyl acetate (gradient elution from 50:1 to 3:2 v/v) was used as the eluent for chromatography.

MS calcd for (C₃₀H₄₀N₄O₅S+H)⁺: 569

MS found (electrospray): (M+H)⁺=569

Intermediate 26
2-Amino-3-(1,3-thiazol-2-yl)propanoic acid, tert-butyl ester

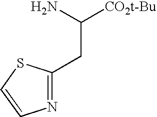

70% Aqueous perchloric acid (1.47 mL) was added dropwise to a stirred suspension of 2-amino-3-(1,3-thiazol-2-yl)propanoic acid (1.077 g, 6.25 mmol) in tert-butyl acetate (34 mL). The suspension was stirred under nitrogen at room temperature overnight. Ethyl acetate (100 mL) was added and the mixture was basified to ˜pH 8 with saturated sodium hydrogen carbonate solution and solid sodium hydrogen carbonate. The organic solution was separated, dried over magnesium sulfate and concentrated to give the title compound as a colourless oil.

¹H NMR (CDCl₃): δ 7.75 (d, 1H), 7.27 (d, 1H), 3.88 (dd, 1H), 3.50 (dd, 1H), 3.32 (dd, 1H), 1.47 (s, 9H).

Intermediate 27
2-[[N-(5-Methyl-1,3-thiazol-2-yl)methylene]amino]-3-(1,3-thiazol-2-yl)propanoic acid, tert-butyl ester

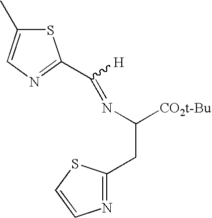

The title compound was prepared in a similar manner to Intermediate 3, using Intermediate 26 in place of Intermediate 2.

¹H NMR (CDCl₃): δ 8.28 (s, 1H), 7.69 (d, 1H), 7.60 (d, 1H), 7.19 (d, 1H), 4.46 (m, 1H), 3.75 (dd, 1H), 3.58 (dd, 1H), 2.50 (s, 3H) and 1.42 (s, 9H).

Intermediate 28
rel-(2R,4S,5R)-5-(5-Methyl-1,3-thiazol-2-yl)-2-(1,3-thiazol-2-ylmethyl)pyrrolidine-2,4-dicarboxylic acid, 2-tert-butyl ester, 4-methyl ester

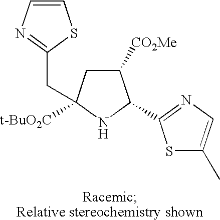

The title compound was prepared in a similar manner to Intermediate 4, using Intermediate 27 in place of Intermediate 3.

MS calcd for (C₁₉H₂₅N₃O₄S₂+H)⁺: 424

MS found (electrospray): (M+H)⁺=424

Intermediate 29
rel-(2R,4S,5R)-1-(3-Methoxy-4-tert-butylbenzoyl)-5-(5-methyl-1,3-thiazol-2-yl)-2-(1,3-thiazol-2-ylmethyl)pyrrolidine-2,4-dicarboxylic acid, 2-tert-butyl ester,

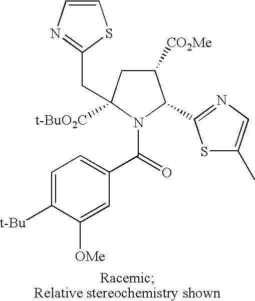

The title compound was prepared in a similar manner to Intermediate 5, using Intermediate 28 in place of Intermediate 4.

MS calcd for (C₃₁H₃₉N₃O₆S₂+H)⁺: 614

MS found (electrospray): (M+H)⁺=614

Intermediate 30
rel-(2R,4S,5R)-4-(Hydroxymethyl)-1-(3-methoxy-4-tert-butylbenzoyl)-5-(5-methyl-1,3-thiazol-2-yl)-2-(1,3-thiazol-2-ylmethyl)pyrrolidine-2-carboxylic acid, tert-butyl ester

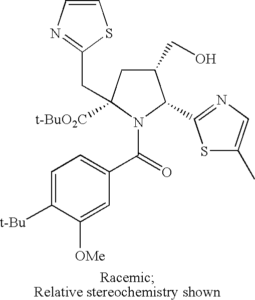

The title compound was prepared in a similar manner to Intermediate 6, using Intermediate 29 in place of Intermediate 5.

MS calcd for (C₃₀H₃₉N₃O₅S₂+H)⁺: 586

MS found (electrospray): (M+H)⁺=586

Intermediate 31
rel-(2R,4S,5R)-1-(3-Methoxy-4-tert-butylbenzoyl)-4-(methoxymethyl)-5-(5-methyl-1,3-thiazol-2-yl)-2-(1,3-thiazol-2-ylmethyl)pyrrolidine-2-carboxylic acid, tert-butyl ester

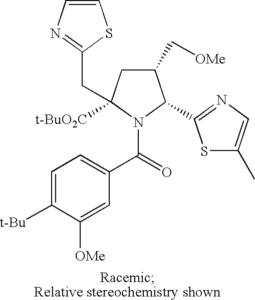

The title compound was prepared in a similar manner to Intermediate 7, using Intermediate 30 in place of Intermediate 6.

MS calcd for (C₃₁H₄₁N₃O₅S₂+H)⁺: 600

MS found (electrospray): (M+H)⁺=600

Intermediate 32
2-[[N-(5-Methyl-1,3-thiazol-2-yl)methylene]amino]-3-(1,2-thiazol-3-yl)propanoic acid, tert-butyl ester

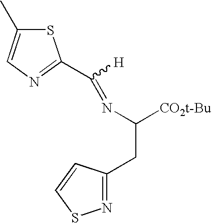

The title compound was prepared in a similar manner to Intermediate 3, using Intermediate 14 in place of Intermediate 2.

¹H NMR (CDCl₃): δ 8.53 (d, 1H), 8.23 (d, 1H), 7.56 (d, 1H), 7.08 (d, 1H), 4.50 (dd, 1H), 3.57 (dd, 1H), 3.39 (dd, 1H), 2.50 (d, 3H) and 1.44 (s, 9H).

Intermediate 33
rel-(2R,4S,5R)-5-(5-Methyl-1,3-thiazol-2-yl)-2-(1,2-thiazol-3-ylmethyl)pyrrolidine-2,4-dicarboxylic acid, 2-tert-butyl ester, 4-methyl ester

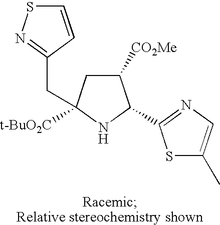

The title compound was prepared in a similar manner to Intermediate 4, using Intermediate 32 in place of Intermediate 3.

MS calcd for (C₁₉H₂₅N₃O₄S₂+H)⁺: 424

MS found (electrospray): (M+H)⁺=424

Intermediate 34
rel-(2R,4S,5R)-1-(3-Methoxy-4-tert-butylbenzoyl)-5-(5-methyl-1,3-thiazol-2-yl)-2-(1,2-thiazol-3-ylmethyl)pyrrolidine-2,4-dicarboxylic acid, 2-tert-butyl ester, 4-methyl ester

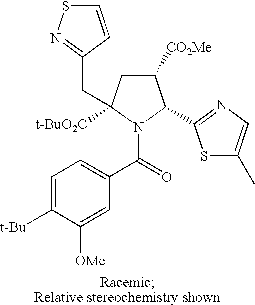

The title compound was prepared in a similar manner to Intermediate 5, using Intermediate 33 in place of Intermediate 4.

MS calcd for (C₃₁H₃₉N₃O₆S₂+H)⁺: 614

MS found (electrospray): (M+H)⁺=614

Intermediate 35
rel-(2R,4S,5R)-4-(Hydroxymethyl)-1-(3-methoxy-4-tert-butylbenzoyl)-5-(5-methyl-1,3-thiazol-2-yl)-2-(1,2-thiazol-3-ylmethyl)pyrrolidine-2-carboxylic acid, tert-butyl ester

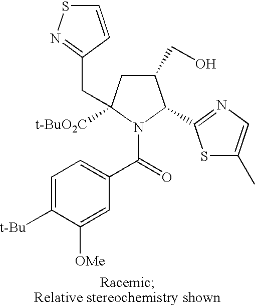

The title compound was prepared in a similar manner to Intermediate 6, using Intermediate 34 in place of Intermediate 5.

MS calcd for (C₃₀H₃₉N₃O₅S₂+H)⁺: 586

MS found (electrospray): (M+H)⁺=586

Intermediate 36
rel-(2R,4S,5R)-1-(3-Methoxy-4-tert-butylbenzoyl)-4-(methoxymethyl)-5-(5-methyl-1,3-thiazol-2-yl)-2-(1,2-thiazol-3-ylmethyl)pyrrolidine-2-carboxylic acid, tert-butyl ester

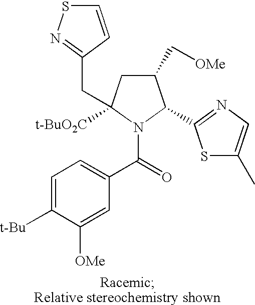

The title compound was prepared in a similar manner to Intermediate 7, using Intermediate 35 in place of Intermediate 6.

MS calcd for (C₃₁H₄₁N₃O₅S₂+H)⁺: 600

MS found (electrospray): (M+H)⁺=600

Intermediate 37
2-[[N-(5-Methyl-1,3-thiazol-2-yl)methylene]amino]-3-(1H-pyrazol-1-yl)propanoic acid, tert-butyl ester

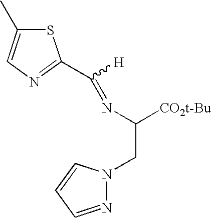

The title compound was prepared in a similar manner to Intermediate 3, using Intermediate 20 in place of Intermediate 2.

¹H NMR (CDCl₃): δ 7.98 (s, 1H), 7.56 (bd, 1H), 7.50 (bd, 1H), 7.34 (d, 1H), 6.13 (t, 1H), 4.82-4.73 (m, 1H), 4.50-4.43 (m, 2H), 2.51 (s, 3H) and 1.47 (s, 9H).

Intermediate 38
rel-(2R,4S,5R)-5-(5-Methyl-1,3-thiazol-2-yl)-2-(1H-pyrazol-1-ylmethyl)pyrrolidine-2,4-dicarboxylic acid, 2-tert-butyl ester, 4-methyl ester

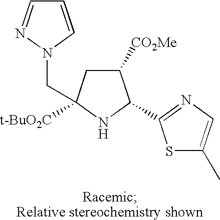

The title compound was prepared in a similar manner to Intermediate 4, using Intermediate 37 in place of Intermediate 3.

MS calcd for (C₁₉H₂₆N₄O₄S+H)⁺: 407

MS found (electrospray): (M+H)⁺=407

Intermediate 39
rel-(2R,4S,5R)-1-(3-Methoxy-4-tert-butylbenzoyl-5-(5-methyl-1,3-thiazol-2-yl)-2-(1H-pyrazol-1-ylmethyl)pyrrolidine-2,4-dicarboxylic acid, 2-tert-butyl ester, 4-methyl ester

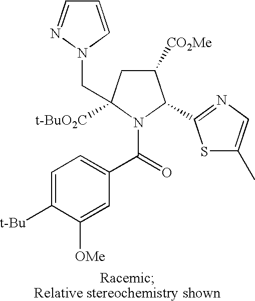

The title compound was prepared in a similar manner to Intermediate 5, using Intermediate 38 in place of Intermediate 4.

MS calcd for (C₃₁H₄₀N₄O₆S+H)⁺: 597

MS found (electrospray): (M+H)⁺=597

Intermediate 40
rel-(2R,4S,5R)-4-(Hydroxymethyl)-1-(3-methoxy-4-tert-butylbenzoyl)-5-(5-methyl-1,3-thiazol-2-yl)-2-(1H-pyrazol-1-ylmethyl)pyrrolidine-2,4-dicarboxylic acid, 2-tert-butyl ester

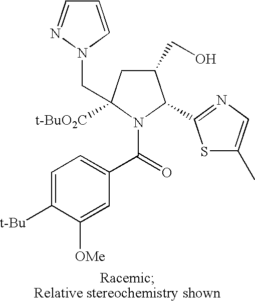

The title compound was prepared in a similar manner to Intermediate 6, using Intermediate 39 in place of Intermediate 5.

MS calcd for (C₂₉H₃₈N₄O₅S+H)⁺: 569

MS found (electrospray): (M+H)⁺=569

Intermediate 41
rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2,4-dicarboxylic acid, 2-tert-butyl ester

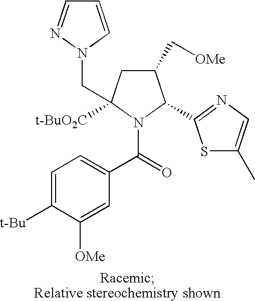

The title compound was prepared in a similar manner to Intermediate 7, using Intermediate 40 in place of Intermediate 6.

MS calcd for (C₃₁H₄₂N₄O₅S+H)⁺: 583

MS found (electrospray): (M+H)⁺=583

### Example 1

rel-(2R,4S,5R)-1-(3-Methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid

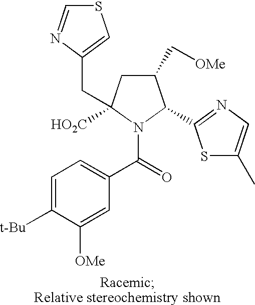

A solution of rel-(2R,4S,5R)-1-(3-methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid, tert-butyl ester (Intermediate 7, 0.205 g, 0.34 mmol) in dichloromethane (3 mL) was treated with trifluoroacetic acid (3 mL) at room temperature for 4 hours. The mixture was evaporated and the residue was triturated with diethyl ether to give the title compound as a white solid.

MS calcd for (C₂₇H₃₃N₃O₅S₂+H)⁺: 544

MS found (electrospray): (M+H)⁺=544

¹H NMR (CD₃OD): δ 9.09 (d, 1H), 8.31 (d, 1H), 7.53 (d, 1H), 7.51 (d, 1H), 7.21 (d, 1H), 6.79 (dd, 1H), 6.55 (d, 1H), 5.05 (d, 1H), 4.07 (d, 1H), 3.70 (s, 3H), 3.55 (d, 1H), 2.98 (s, 3H), 2.95 (dd, 1H), 2.50 (t, 1H), 2.40 (dd, 1H), 2.36 (d, 3H), 2.13 (t, 1H) and 1.32 (s, 9H).

### Example 2

Enantiomer A of rel-(2R,4S,5R)-1-(3-Methoxy-4-tert-butylbenzoyl)-4-(methoxy-methyl)-5-(5-methyl-1,3-thiazol-2-yl)-2-(1,3-thiazol-4-ylmethyl)pyrrolidine-2-carboxylic acid

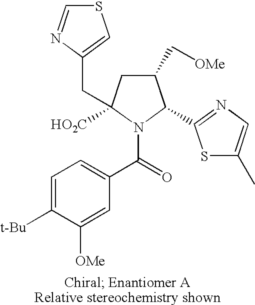

rel-(2R,4S,5R)-1-(3-methoxy-4-tert-butylbenzoyl)-4-(methoxymethyl)-5-(5-methyl-1,3-thiazol-2-yl)-2-(1,3-thiazol-4-ylmethyl)pyrrolidine-2-carboxylic acid (Example 1) was resolved by preparative chiral HPLC on a Chiralpak AD column using heptane-ethanol (75:25 v/v) containing 0.1% trifluoroacetic acid as eluent to give the first and second eluting enantiomers. The second eluting enantiomer was triturated with diethyl ether to give the title compound.

MS calcd for (C₂₇H₃₃N₃O₅S₂+H)⁺: 544

MS found (electrospray): (M+H)⁺=544

¹H NMR (CD₃OD): δ 9.09 (d, 1H), 8.31 (d, 1H), 7.53 (d, 1H), 7.51 (d, 1H), 7.21 (d, 1H), 6.79 (dd, 1H), 6.55 (d, 1H), 5.05 (d, 1H), 4.07 (d, 1H), 3.70 (s, 3H), 3.55 (d, 1H), 2.98 (s, 3H), 2.95 (dd, 1H), 2.50 (t, 1H), 2.40 (dd, 1H), 2.36 (d, 3H), 2.13 (t, 1H) and 1.32 (s, 9H).

### Example 3

rel-(2R,4S,5R)-1-(3-Methoxy-4-tert-butylbenzoyl)-4-(methoxymethyl)-5-(1,3-thiazol-2-yl)-2-(1,3-thiazol-4-ylmethyl)pyrrolidine-2-carboxylic acid

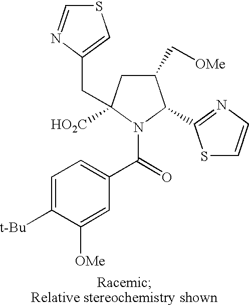

This compound was prepared in a similar manner to Example 1, using Intermediate 12 in place of Intermediate 7 and was purified by column chromatography on silica gel eluting initially with cyclohexane-ethyl acetate (gradient elution from 5:2 to 2:3 v/v) followed by further elution with dichloromethane, then dichloromethane-methanol (gradient elution from 60:1 to 19:1) to give the title compound.

MS calcd for (C₂₆H₃₁N₃O₅S₂+H)⁺: 530

MS found (electrospray): (M+H)⁺=530

¹H NMR (CDCl₃): δ 8.87(d, 1H), 7.79 (d, 1H), 7.25 (dd, 2H), 7.10 (d, 1H), 6.61 (dd, 1H), 6.40 (d, 1H), 5.18 (d, 1H), 4.22 (d, 1H), 3.70 (d, 1H), 3.62 (s, 3H), 3.11 (dd, 1H), 3.01 (s, 3H), 2.48 (dd, 1H), 2.31 (m, 1H), 2.16 (m, 2H), 1.89 (br, 1H) and 1.30 (s, 9H).

### Example 4

rel-(2R,4S,5R)-1-(3-Methoxy-4-tert-butylbenzoyl)-4-(methoxymethyl)-5-(1,3-thiazol-2-yl)-2-(1,2-thiazol-3-ylmethyl)pyrrolidine-2-carboxylic acid

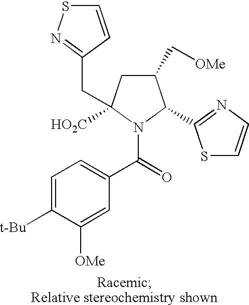

The compound was prepared in a similar manner to Example 1, using Intermediate 19 in neat trifluoroacetic acid in place of Intermediate 7 in trifluoroacetic acid and dichloromethane, and re-evaporating from dichloromethane in place of triturating with diethyl ether. Purification by reverse phase HPLC on a C₁₈ column, using a two-solvent gradient elution with (A) water containing formic acid (0.1%) and (B) acetonitrile-water (95:5 v/v) containing formic acid (0.05%) as the eluents, and analysis of the fractions by electrospray mass spectroscopy provided the title compound.

MS calcd for (C₂₆H₃₁N₃O₅S₂+H)⁺: 530

MS found (electrospray): (M+H)⁺=530

¹H NMR (CDCl₃): δ 8.70 (1H, d), 7.78 (1H, d), 7.31 (1H, d), 7.27 (1H, d), 7.12 (1H, d), 6.63 (1H, dd), 6.45 (1H, d), 5.19 (1H, d), 4.22 (1H, d), 3.73 (1H, d), 3.64 (3H, s), 3.08 (1H, dd), 3.00 (3H, s), 2.42 (1H, dd), 2.31 (1H, t), 2.17 (1H, t), 2.04-1.94 (1H, m) and 1.29 (9H, s). Acid proton not seen.

### Example 5

Enantiomer A of rel-(2R,4S,5R)-1-(3-Methoxy-4-tert-butylbenzoyl)-4-(methoxy-methyl)-5-(1,3-thiazol-2-yl)-2-(1,2-thiazol-3-ylmethyl)pyrrolidine-2-carboxylic acid

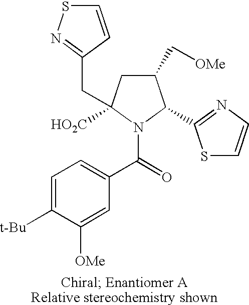

rel-(2R,4S,5R)-1-(3-Methoxy-4-tert-butylbenzoyl)-4-(methoxymethyl)-5-(1,3-thiazol-2-yl)-2-(1,2-thiazol-3-ylmethyl)pyrrolidine-2-carboxylic acid (Example 4) was resolved by preparative chiral HPLC on a Chiralpak AD column using heptane-ethanol (85:15 v/v) containing 0.1% trifluoroacetic acid as eluent to give the first and second eluting enantiomers. The second eluting enantiomer was dissolved in dichloromethane, washed with sodium hydrogen carbonate solution; dried (hydrophobic frit) and solvent removed to give the title compound.

MS calcd for (C₂₆H₃₁N₃O₅S₂+H)⁺: 530

MS found (electrospray): (M+H)⁺=530

¹NMR (CDCl₃): δ 8.70 (d, 1H), 7.78 (d, 1H), 7.31 (d, 1H), 7.27 (d, 1H), 7.12 (d, 1H), 6.63 (dd, 1H), 6.45 (d, 1H), 5.19 (d, 1H), 4.23 (d, 1H), 3.74 (d, 1H), 3.64 (s, 3H), 3.08 (dd, 1H), 3.00 (s, 3H), 2.43 (dd, 1H), 2.31 (t, 1H), 2.17 (t, 1H), 2.06-1.94 (m, 1H), 1.29 (s, 9H). Acid proton not seen.

### Example 6

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid

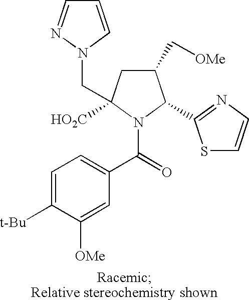

The compound was prepared in a similar manner to Example 1, using Intermediate 25 in place of Intermediate 7. Purification by column chromatography on silica gel eluting initially with cyclohexane-ethyl acetate (gradient elution from 50:1 v/v to 1:9 v/v) followed by further elution with dichloromethane-methanol (9:1 v/v) gave the title compound.

MS calcd for (C₂₆H₃₂N₄O₅S+H)⁺: 513

MS found (electrospray): (M+H)⁺=513

¹H NMR (CD₃OD): δ 7.81 (m, 2H), 7.66 (d, 1H), 7.56 (d, 1H), 7.17 (d, 1H), 6.71 (dd, 1H), 6.62 (d, 1H), 6.42 (m, 1H), 5.20 (m, 2H), 4.83 (m, 1H), 3.70 (s, 3H), 2.96 (s, 3H), 2.91 (m, 1H), 2.45 (m, 2H), 2.14 (t, 1H), 1.49 (m, 1H) and 1.31 (s, 9H).

### Example 7

(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid

[Enantiomer A of rel-(2R,4S,5R)-1-(3-Methoxy-4-tert-butylbenzoyl)4-(methoxy-methyl)-2-(1H-pyrazol-1-ylmethyl)-5-(1,3-thiazol-2-yl)pyrrolidine-2-carboxylic acid]

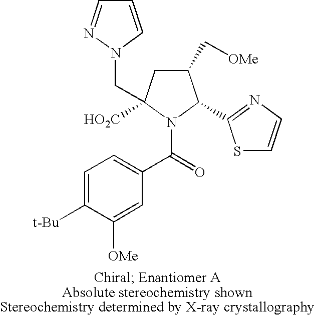

Alternative Method A

rel-(2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid (Example 6) was resolved by preparative chiral HPLC on a Sumichiral OA4900 column using heptane-ethanol (70:30 v/v) containing 0.1% trifluoroacetic acid as eluent to give the first and second eluting enantiomers. The second eluting enantiomer was dissolved in dichloromethane, washed with water (×4), washed with brine, and then dried (sodium sulfate) and solvent removed to give the title compound.

MS calcd for (C₂₆H₃₂N₄O₅S+H)⁺: 513

MS found (electrospray): (M+H)⁺=513

¹H NMR (CD₃OD): δ 7.81 (m, 2H), 7.66 (d, 1H), 7.57 (d, 1H), 7.17 (d, 1H), 6.70 (dd, 1H), 6.61 (d, 1H), 6.42 (m, 1H), 5.20 (m, 2H), 4.85 (m, 1H), 3.70 (s, 3H), 2.97 (s, 3H), 2.91 (m, 1H), 2.44 (m, 2H), 2.15 (t, 1H), 1.47 (m, 1H) and 1.31 (s, 9H).

Alternative Method B

Part 1

rel-(2R,4S,5R)-1-(3-Methoxy-4-tert-butylbenzoyl)-4-(methoxy-methyl)-2-(1H-pyrazol-1-ylmethyl)-5-(1,3-thiazol-2-yl)pyrrolidine-2-carboxylic acid, 2-tert-butyl ester (prepared in a similar manner to that described in Intermediate 25; 1.95 g) was resolved by preparative chiral HPLC on a Chiralpak AD column using heptane-isopropanol (90:10 v/v) as eluent. Fractions containing the first eluting enantiomer (retention time 7.25 minutes) were dissolved in dichloromethane, washed with saturated aqueous sodium bicarbonate solution and the dichloromethane evaporated to afford Enantiomer A (0.76 g), used directly in Part 2, below. The slower eluting enantiomer (retention time 10 minutes) was not required further.

The chiral HPLC resolution was repeated in a similar manner using an additional aliquot of rel-(2R,4S,5R)-1-(3-Methoxy-4-tert-butylbenzoyl)-4-(methoxy-methyl)-2-(1H-pyrazol-1-ylmethyl)-5-(1,3-thiazol-2-yl)pyrrolidine-2-carboxylic acid, 2-tert-butyl ester (1.55 g), to afford an additional batch of the fast eluting Enantiomer A (0.55 g), used directly in Part 2, below.

Part 2

The two combined batches of Enantiomer A of rel-(2R,4S,5R)-1-(3-methoxy-4-tert-butylbenzoyl)-4-(methoxy-methyl)-2-(1H-pyrazol-1-ylmethyl)-5-(1,3-thiazol-2-yl)-pyrrolidine-2-carboxylic acid, 2-tert-butyl ester (Part 1 above; combined 1.31 g, 2.32 mmol) were dissolved in trifluoroacetic acid (20 mL) and the resulting solution stirred at room temperature for 3 hours. The mixture was evaporated and the residue partitioned between dichloromethane and saturated aqueous sodium bicarbonate solution. The aqueous phase was removed using a hydrophobic frit and the organic solution evaporated to afford a gum. This was dissolved in diethyl ether in a stoppered flask and allowed to crystallize slowly overnight. The crystals were filtered, washed with a small quantity of diethyl ether and dried in vacuo to afford the title compound.

MS calcd for (C₂₆H₃₂N₄O₅S+H)⁺: 513

MS found (electrospray): (M+H)⁺=513

¹H NMR (CD₃OD): δ 7.83 (m, 2H), 7.66 (d, 1H), 7.57 (d, 1H), 7.19 (d, 1H), 6.72 (dd, 1H), 6.63 (d, 1H), 6.45 (t, 1H), 5.21 (m, 2H), 4.86 (m, 1H), 3.70 (s, 3H), 2.97 (s, 3H), 2.94 (m, 1H), 2.44 (m, 2H), 2.16 (t, 1H), 1.48 (m, 1H) and 1.31 (s, 9H).

The absolute stereochemistry of this compound was determined by X-ray crystallography and shown to be (2R,4S,5R), as drawn.

### Example 8

rel-(2R,4S,5R)-1-(3-Methoxy-4-tert-butylbenzoyl)-4-(methoxymethyl)-5-(5-methyl-1,3-thiazol-2-yl)-2-(1,3-thiazol-2-ylmethyl)pyrrolidine-2-carboxylic acid

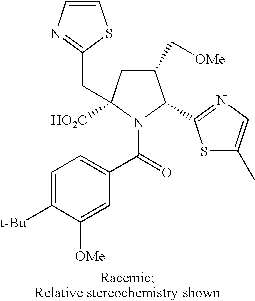

The compound was prepared in a similar manner to Example 1, using Intermediate 31 in place of Intermediate 7. The impure product was dissolved in dichloromethane and washed with sodium hydrogen carbonate solution, dried (hydrophobic frit) and the solvent removed. The residue was triturated with diethyl ether to give the title compound.

MS calcd for (C₂₇H₃₃N₃O₅S₂+H)⁺: 544

MS found (electrospray): (M+H)⁺=544

¹H NMR (CDCl₃): δ 7.85 (d, 1H), 7.42 (d, 1H), 7.38 (d, 1H), 7.15 (d, 1H), 6.80 (dd, 1H), 6.43 (d, 1H), 5.08 (d, 1H), 4.34 (d, 1H), 3.95 (d, 1H), 3.64 (s, 3H), 3.10 (dd,. 1H), 3.01 (dd, 1H), 3.01 (s, 3H), 2.49 (dd, 1H), 2.43-2.33 (m, 4H), 2.13 (t, 1H), 1.92-1.80 (m, 1H) and 1.31 (s, 9H).

### Example 9

rel-(2R,4S,5R)-1-(3-Methoxy-4-tert-butylbenzoyl)-4-(methoxymethyl)-5-(5-methyl-1,3-thiazol-2-yl)-2-(1,2-thiazol-3-ylmethyl)pyrrolidine-2-carboxylic acid

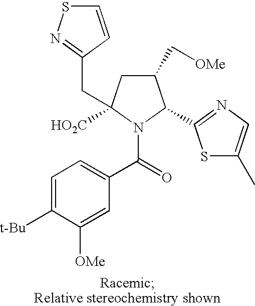

The compound was prepared in a similar manner to Example 1, using Intermediate 36 in neat trifluoroacetic acid in place of Intermediate 7 in trifluoroacetic acid and dichloromethane. The impure product was purified by reverse phase HPLC on a C₁₈ column, using a two-solvent gradient elution with (A) water containing formic acid (0.1%) and (B) acetonitrile-water (95:5 v/v) containing formic acid (0.05%) as the eluents. Analysis of the fractions by electrospray mass spectroscopy provided the title compound.

MS calcd for (C₂₇H₃₃N₃O₅S₂+H)⁺: 544

MS found (electrospray): (M+H)⁺=544

¹H NMR (CDCl₃): δ 8.69 (d, 1H), 7.41 (s, 1H), 7.30 (d, 1H), 7.14 (d, 1H), 6.66 (dd, 1H), 6.42 (d, 1H), 5.04 (d, 1H), 4.22 (d, 1H), 3.72 (d, 1H), 3.65 (s, 3H), 3.08 (dd, 1H), 3.02 (s, 3H), 2.46-2.39 (m, 2H), 2.36 (s, 3H), 2.14 (t, 1H), 1.99-1.88 (m, 1H) and 1.31(s, 9H). Acid proton not seen

### Example 10

rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid

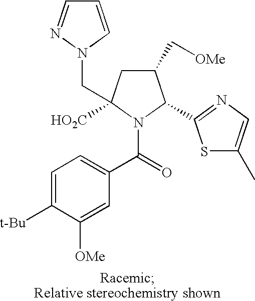

The compound was prepared in a similar manner to Example 1, using Intermediate 41 in place of Intermediate 7. The impure product was purified by reverse phase HPLC on a C₁₈ column, using a two-solvent gradient elution with (A) water containing formic acid (0.1%) and (B) acetonitrile-water (95:5 v/v) containing formic acid (0.05%) as the eluents. Analysis of the fractions by electrospray mass spectroscopy provided the title compound.

MS calcd for (C₂₇H₃₄N₄O₅S+H)⁺: 527

MS found (electrospray): (M+H)⁺=527

¹H NMR (CDCl₃): δ 7.62 (d, 1H), 7.54 (d, 1H), 7.41 (d, 1H), 7.16 (d, 1H), 6.66 (dd, 1H), 6.41 (d, 1H), 6.38 (t, 1H), 5.31 (d, 1H), 5.03 (d, 1H), 4.92 (d, 1H), 3.65 (s, 3H), 3.11 (dd, 1H), 3.04 (s, 3H), 2.54 (dd, 1H), 2.41 (t, 1H), 2.36 (s, 3H), 2.11 (t, 1H), 1.84-1.73 (m, 1H) and 1.31 (s, 9H). Acid proton not seen.

### Example 11

Enantiomer A of re/-(2R,4S,5R)-1-(3-Methoxy-4-tert-butylbenzoyl)-4-(methoxy-methyl)-5-(5-methyl-1,3-thiazol-2-yl)-2-(1H-pyrazol-1-ylmethyl)pyrrolidine-2-carboxylic acid

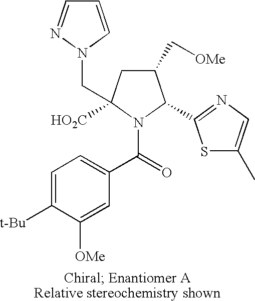

rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid (Example 10) was resolved by preparative chiral HPLC on a Chiralpak AD column using heptane-ethanol (80:20 v/v) containing 0.1% trifluoroacetic acid as eluent to give the first and second eluting enantiomers. The second eluting enantiomer was dissolved in dichloromethane, washed with sodium hydrogen carbonate solution; dried (hydrophobic frit) and solvent removed to give the title compound.

MS calcd for (C₂₇H₃₄N₄O₅S +H)⁺: 527

MS found (electrospray): (M+H)⁺=527

¹H NMR (CDCl₃): δ 7.62 (d, 1H), 7.54 (d, 1H), 7.41 (d, 1H), 7.15 (d, 1H), 6.66 (dd, 1H), 6.40 (d, 1H), 6.38 (t, 1H), 5.31 (d, 1H), 5.03 (d, 1H), 4.92 (d, 1H), 3.64 (s, 3H), 3.11 (dd, 1H), 3.04 (s, 3H), 2.54 (dd, 1H), 2.40 (t, 1H), 2.36 (d, 3H), 2.11 (t, 1H), 1.83-1.72 (m, 1H) and 1.31 (s, 9H). Acid proton not seen.

### Example 12

Enantiomer A of rel-(2R,4S,5R)-1-(3-Methoxy4-tert-butylbenzoyl)4-(methoxy-methyl)-5-(1,3-thiazol-2-yl)-2-(1,3-thiazol-4-ylmethyl)pyrrolidine-2-carboxylic acid

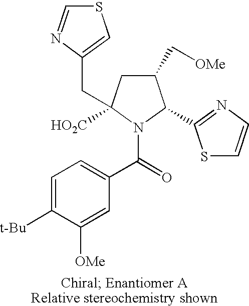

rel-(2R,4S,5R)-1-(3-Methoxy-4-tert-butylbenzoyl)-4-(methoxy-methyl)-5-(1,3-thiazol-2-yl)-2-(1,3-thiazol-4-ylmethyl)pyrrolidine-2-carboxylic acid (prepared in a similar manner to that described in Example 3; 0.530 g) was resolved by preparative chiral HPLC on a Sumichiral OA-4900 column, using heptane-ethanol (65:35 v/v) containing 0.1% trifluoroacetic acid as eluent to give the first and second eluting enantiomers. The second eluting enantiomer was partitioned between dichloromethane and saturated aqueous sodium bicarbonate solution. The dichloromethane solution was separated using a hydrophobic frit and evaporated in vacuo to afford the title compound, a foam.

MS calcd for (C₂₆H₃₁N₃O₅S₂+H)⁺: 530

MS found (electrospray): (M+H)⁺=530

¹H NMR (CDCl₃): δ 14.59 (1H, s), 8.88 (1H, s), 7.80 (1H, d), 7.29-7.28 (2H, partly obscured by chloroform signal), 7.12 (1H, d), 6.63 (1H, d), 6.42 (1H, s), 5.17 (1H, d), 4.24 (1H, d), 3.73 (1H, d), 3.63 (3H, s), 3.16-3.08 (1H, m), 3.01 (3H, s), 2.55-2.45 (1H, m), 2.36-2.26 (1H, m), 2.24-2.10 (2H, m) and 1.30 (9H, s).

The compounds according to the invention may be formulated for administration in any convenient way, and the invention therefore also includes within its scope pharmaceutical compositions for use in therapy, comprising a compound of formula (Ia) or a physiologically acceptable salt or solvate thereof in admixture with one or more physiologically acceptable diluents or carriers.

The compounds of the present invention can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (Ia) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (Ia). A topical formulation contains suitably 0.01 to 5.0% of a compound of Formula (Ia). The active ingredient may be administered from 1 to 6 times per day, preferably once, sufficient to exhibit the desired activity, as is readily apparent to one skilled in the art.

Compositions of Formula (Ia) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional non-CFC propellant such as 1,1,1,2-tetrafluoroethane or 1,1,1,2,3,3,3-heptafluoropropane.

A typical suppository formulation comprises a compound of Formula (Ia) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxological effects are expected when compounds of the present invention are administered in accordance with the present invention.

### Assays

The potential for chemical entities of the invention to inhibit NS5B wildtype HCV polymerase activity, genotype la and genotype lb, may be demonstrated, for example, using the following in vitro assays:

In Vitro Detection of inhibitors of HCV RNA-Dependent RNA Polymerase Activity

Incorporation of [³³P]-GMP into RNA was followed by absorption of the biotin labelled RNA polymer by streptavidin containing SPA beads. A synthetic template consisting of biotinylated 13mer-oligoG hybridised to polyrC was used as a homopolymer substrate.

a) Genotype 1a C-Terminally Truncated (delta21) Enzyme

HCV RNA Polymerase [Recombinant NS5B with C-terminal 21 amino acid deletion and C-terminal 6His-tag (Ferrari et al. J. Virol. 73(2), 1999, 1649. ‘Characterization of soluble hepatitis C virus RNA-dependent RNA polymerase expressed in Escherichia coli.’) expressed in E. coli and purified to homogeneity] was added to 25 nM final concentration. Polymerase of genotype la was from strain H77 (Yanagi, M., Purcell, R. H., Emerson, S. U. & Bukh, J. (1997), Proceedings of the National Academy of Sciences, USA 94, 8738-8743) containing a sequence change from valine to isoleucine at position 180.

Reaction Conditions were 25 nM enzyme, 1.5 μg/ml oligo-rG13/poly-rC and 0.2 μCi α-³³P-GTP in 0.5 μM GTP (20 Ci/mMol) , 20 mM Tris pH 7.5, 23 mM NaCl, 3 mM DTT, 5 mM MgCl₂, 1 mM MnCl₂.

Enzyme was diluted to 500 nM concentration in 20 mM Tris-HCl, pH 7.5, 25 mM NaCl and 3 mM DTT.

4× concentrated assay buffer mix was prepared using 1M Tris-HCl, pH7.5 (1 mL), 5M NaCl (0.25 mL), 1M DTT (0.12 mL) and Water (8.63 mL), Total 10 mL.

2× concentrated first reagent was prepared using 4× concentrated assay buffer mix (5 μL), 40 μ/μL RNasin (0.1 μL), 20 μg/mL polyrC/biotinylated-oligorG (1.6 μL), 500 nM enzyme (1 μL ) and Water (2.3 μL), Total 10 μL/well.

2× concentrated second reagent was prepared using 1 M MgCl₂ (0.1 EL), 1M MnCl₂ (0.02 μL), 25 μM GTP (0.4 μL), α-[³³P]-GTP (10 μCi/μL, 0.02 μL) and water (9.5 μL), Total 10 μL/well.

The assay was set up using compound (1 μL in 100% DMSO), first reagent (10 μL), and second reagent (10 μL), Total 21 μL.

The reaction was performed in a U-bottomed, white, 96-well plate. The reaction was mixed on a plate-shaker, after addition of the Enzyme, and incubated for 1 h at 22° C. After this time, the reaction was stopped by addition of 60 μL 1.5 mg/ml streptavidin SPA beads (Amersham) in 0.1 M EDTA in PBS. The beads were incubated with the reaction mixture for 1 h at 22° C. after which 100 μL 0.1 M EDTA in PBS was added. The plate was sealed, mixed centrifuged and incorporated radioactivity determined by counting in a Trilux (Wallac) or Topcount (Packard) Scintillation Counter.

After subtraction of background levels without enzyme, any reduction in the amount of radioactivity incorporated in the presence of a compound, compared to that in the absence, was taken as a measure of the level of inhibition. Ten concentrations of compounds were tested in three- or fivefold dilutions. From the counts per minute, percentage of inhibition at highest concentration tested or IC₅₀s for the compounds were calculated using GraFit 3, GraFit 4 or GraFit 5 (Erithacus Software Ltd.) software packages or a data evaluation macro for Excel based on XLFit software (IDBS).

b) Genotype 1b Full-Length Enzyme

Reaction Conditions were 0.5 μM [³³P]-GTP (20 Ci/mMol), 1 mM Dithiothreitol, 20 mM MgCl₂, 5 mM MnCl₂, 20 mM Tris-HCl, pH7.5, 1.6 μg/mL polyC/0.256 μM biotinylated oligoG13, 10% glycerol, 0.01% NP-40, 0.2 u/μL RNasin and 50 mM NaCl.

HCV RNA Polymerase (Recombinant full-length NS5B (Lohmann et al, J. Virol. 71 (11), 1997, 8416. ‘Biochemical properties of hepatitis C virus NS5B RNA-dependent RNA polymerase and identification of amino acid sequence motifs essential for enzymatic activity’) expressed in baculovirus and purified to homogeneity) was added to 4 nM final concentration.

5× concentrated assay buffer mix was prepared using 1M MnCl₂ (0.25 mL), glycerol (2.5 mL), 10% NP-40 (0.025 mL) and Water (7.225 mL), Total 10 mL.

2× concentrated enzyme buffer contained 1M-Tris-HCl, pH7.5 (0.4 mL), 5M NaCl (0.2 mL), 1M-MgCl₂(0.4 mL), glycerol (1 mL), 10% NP-40 (10 μL), 1M DTT (20 μL) and water (7.97 mL), Total 10 mL.

Substrate Mix was prepared using 5x Concentrated assay Buffer mix (4 μL), [³³P]-GTP (10 μCi/μL, 0.02 μL), 25 μM GTP (0.4 μL), 40 μ/μL RNasin (0.1 μL), 20 μg/mL polyrC/biotinylated-oligorG (1.6 μL), and Water (3.94 μL), Total 10 μL.

Enzyme Mix was prepared by adding 1 mg/ml full-length NS5B polymerase (1.5 μL) to 2.81 mL 2×-concentrated enzyme buffer.

The Assay was set up using compound (1μL), Substrate Mix (10 μL), and Enzyme Mix (added last to start reaction) (10 μL), Total 21 μL.

The reaction was performed in a U-bottomed, white, 96-well plate. The reaction was mixed on a plate-shaker, after addition of the Enzyme, and incubated for 1 h at 22° C. After this time, the reaction was stopped by addition of 40 μL 1.875 mg/ml streptavidin SPA beads in 0.1 M EDTA. The beads were incubated with the reaction mixture for 1 h at 22° C. after which 120 μL 0.1 M EDTA in PBS was added. The plate was sealed, mixed centrifuged and incorporated radioactivity determined by counting in a Trilux (Wallac) or Topcount (Packard) Scintillation Counter.

After subtraction of background levels without enzyme, any reduction in the amount of radioactivity incorporated in the presence of a compound, compared to that in the absence, was taken as a measure of the level of inhibition. Ten concentrations of compounds were tested in three- or fivefold dilutions. From the counts, percentage of inhibition at highest concentration tested or IC₅₀s for the compounds were calculated using GraFit 3, GraFit 4 or GraFit 5 (Erithacus Software Ltd.) software packages or a data evaluation macro for Excel based on XLFit software (IDBS).

The potential for compounds of the invention to inhibit NS5B wildtype HCV polymerase activity, genotype la and genotype lb may be demonstrated, for example, using the following cell based assays:

Replicon ELISA Cell Based Assay

Method

100 μL of medium containing 10% FCS were added to each well of clear, flat-bottomed 96 well microplates, excepting wells in the top row. Test compound was diluted in assay medium to twice the final required starting concentration from a 40 mM stock solution in DMSO. 200 μL of the starting dilution were introduced into two wells each in the top row and doubling dilutions made down the plate by the sequential transfer of 100 μL aliquots with thorough mixing in the wells; the final 100 μL were discarded. The two bottom rows were not used for compound dilutions. Huh-7 HCV replicon cell monolayers nearing confluency were stripped from growth flasks with versene-trypsin solution and the cells were resuspended in assay medium at either 2×10⁵ cells/mL (sub-line 5-15; genotype 1b; Lohmann, V., Korner, F., Koch, J-O., Herian, U., Thielmann, L. and Bartenschlager, R., 1999, Science, 285, pp 110-113) or at 3×10⁵ cells/mL (genotype 1a; Gu, B., Gates, A.T., Isken, O., Behrens, S. E. and Sarisky, R. T., J. Virol., 2003, 77, 5352-5359). 100 μL of cell suspension were added to all wells and the plates incubated at 37° C. for 72 hours in a 5% CO₂ atmosphere.

Following incubation, the assay medium was aspirated from the plates. The cell sheets were washed by gentle immersion in phosphate buffered saline (PBS), which was then aspirated off, and fixed with acetone:methanol (1:1) for 5 minutes. Following a further wash with PBS, 100 μL of ELISA diluent (PBS+0.05% v/v Tween 20+2% w/v skimmed milk powder) were added to all wells and the plates incubated at 37° C. for 30 minutes on an orbital platform. The diluent was removed and each well then received 50 μL of a 1/200 dilution of anti-HCV specific, murine, monoclonal antibody (either Virostat #1872 or #1877), except for wells in one of the compound-free control rows which received diluent alone to act as negative controls. The plates were incubated at 37° C. for 2 hours and washed 3 times with PBS/0.05% Tween 20, then 50 μL of horseradish peroxidase conjugated, anti-mouse, rabbit polyclonal serum (Dako #P0260), diluted 1/1000, were added to all wells. The plates were incubated for a further hour, the antibody removed and the cell sheets washed 5 times with PBS/Tween and blotted dry. The assay was developed by the addition of 50 μL of ortho-phenylenediamine/peroxidase substrate in urea/citrate buffer (SigmaFast, Sigma #P-9187) to each well, and colour allowed to develop for up to 15 minutes. The reaction was stopped by the addition of 25 μL per well of 2 M sulphuric acid and the plates were read at 490 nm on a Fluostar Optima spectrophotometer.

The substrate solution was removed and the plates were washed in tap water, blotted dry and the cells stained with 5% carbol fuchsin in water for 30 minutes. The stain was discarded and the cell sheets washed, dried and examined microscopically to assess cytotoxicity.

Data Analysis

The absorbance values from all compound-free wells that had received both primary and secondary antibodies were averaged to obtain a positive control value. The mean absorbance value from the compound-free wells that had not received the primary antibody was used to provide the negative (background) control value. The readings from the duplicate wells at each compound concentration were averaged and, after the subtraction of the mean background from all values, were expressed as a percentage of the positive control signal. The quantifiable and specific reduction of expressed protein detected by the ELISA in the presence of a drug can be used as a measure of replicon inhibition. GraFit software (Erithacus Software Ltd.) was used to plot the curve of percentage inhibition against compound concentration and derive the 50% inhibitory concentration (IC₅₀) for the compound.

Results

| | IC₅₀ in | IC₅₀ in full | | |
|---|---|---|---|---|
| | delta-21 | length 1b | IC₅₀ in 1a | IC₅₀ in 1b |
| | 1a enzyme | enzyme | replicon | replicon |
| | inhibition | inhibition | cell-based | cell-based |
| Compound | assay (μM) | assay (μM) | assay (μM) | assay (μM) |
| Example 1 | * | # | + | @ |
| Example 2 | * | # | + | @ |
| Example 3 | * | # | + | @ |
| Example 4 | * | # | + | @ |
| Example 5 | * | # | + | @ |
| Example 6 | * | # | + | @ |
| Example 7 | * | # | + | @ |
| Example 8 | * | # | + | @@ |
| Example 9 | * | # | + | @ |
| Example 10 | * | # | + | @ |
| Example 11 | * | # | + | @ |
| Example 12 | * | # | + | @ |
| Compound A | *** | ### | ++ | @@ |
| Compound B | *** | ## | ++ | @@ |
| Compound C | *** | ### | ++ | @@¹ |
| Compound D | ** | ## | ++ | @@ |
| Compound E | *** | ## | +++ | @@ |

Activity ranges

| | | Genotype 1a | | | Genotype 1b | |
|---|---|---|---|---|---|---|
| enzyme | * | <0.75 | μM | # | <0.20 | μM |
| | ** | 0.75-1.00 | μM | ## | 0.20-0.50 | μM |
| | *** | >1.00 | μM | ### | >0.50 | μM |
| replicon | + | <10.00 | μM | @ | <0.15 | μM |
| cell-based | ++ | 10.00-100 | μM | @@ | 0.15-10.00 | μM |
| | +++ | >100 | μM | @@@ | >10.00 | μM |

¹this compound was not tested in the 5-15 cell line; instead, the 11-7 cell line (NS2-5B), containing one additional gene was used in this assay, giving comparable results (Lohmann, V. et al, 1999, Science, 285, pp 110-113).

Compound A corresponds to the racemic compound disclosed as Example 11 in WO2004/037818, rel-(2S,4S,5R)-2-isobutyl-1-(3-methoxy-4-tert-butylbenzoyl)-4-methoxymethyl-5-(1,3-thiazol-2-yl)pyrrolidine-2-carboxylic acid.

Compound B corresponds to the enantiomeric compound disclosed as Example 15 in WO2004/037818, (2S,4S,5R)-2-isobutyl-1-(3-methoxy-4-tert-butylbenzoyl)-4-methoxymethyl-5-(1,3-thiazol-2-yl)pyrrolidine-2-carboxylic acid.

Compound C corresponds to the racemic compound disclosed as Example 24 in WO2004/037818, rel-(2S,4S,5R)-2-isobutyl-1-(3-methoxy-4-tert-butylbenzoyl)-4-methoxymethyl-5-(5-methyl-1,3-thiazol-2-yl)pyrrolidine-2-carboxylic acid.

Compound D corresponds to the enantiomeric compound disclosed as Example 25 in WO2004/037818, Enantiomer A of rel-(2S,4S,5R)-2-isobutyl-1-(3-methoxy-4-tert-butylbenzoyl)-4-methoxymethyl-5-(5-methyl-1,3-thiazol-2-yl)pyrrolidine-2-carboxylic acid.

Compound E corresponds to the racemic compound disclosed as Example 33 in WO2004/037818, rel-(2R,4S,5R)-2-benzyl-1-(3-methoxy-4-tert-butylbenzoyl)-4-methoxymethyl-5-(1,3-thiazol-2-yl)-pyrrolidine-2-carboxylic acid.

Compounds A, B, C, D and E may be made according to the processes described in WO2004/037818.

Structures of Compounds A-E are shown below for the avoidance of doubt.

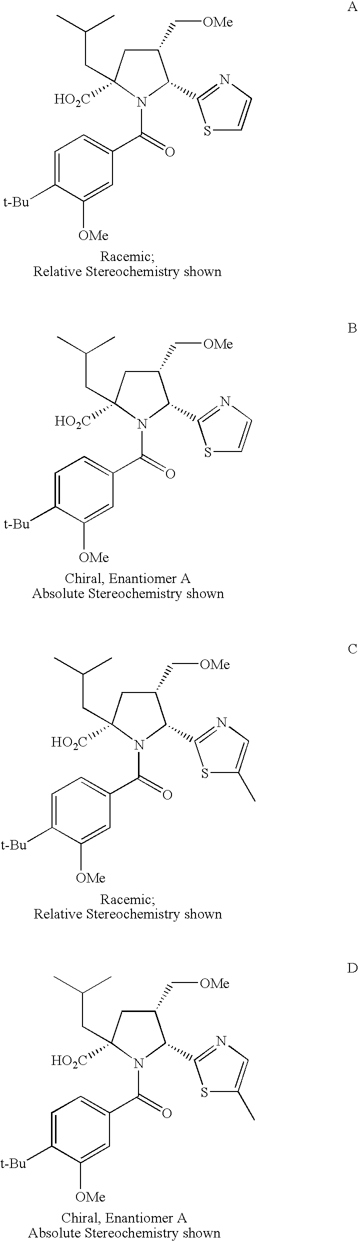

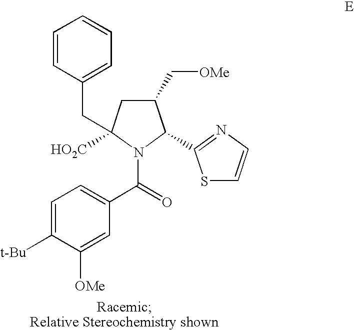

The compounds of the present invention which have been tested demonstrate a surprisingly superior genotype-1a/1b profile, as shown by the IC₅₀ values in the enzyme and cell-based assays across both of the 1a and 1b genotypes of HCV, compared to Compounds A-E. Accordingly, the compounds of the present invention are of great potential therapeutic benefit in the treatment and prophylaxis of HCV.

The pharmaceutical compositions according to the invention may also be used in combination with other therapeutic agents, for example immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

The invention thus provides, in a further aspect, a combination comprising at least one chemical entity chosen from compounds of formula (Ia) and physiologically acceptable salts or solvates thereof, together with at least one other therapeutically active agent.

The combinations referred to above may conveniently be presented for use in the form of a pharmaceutical formulation and thus pharmaceutical formulations comprising a combination as defined above together with at least one pharmaceutically acceptable diluent or carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

All publications, including but not limited to patents and patent applications cited in this specification are herein incorporated by reference as if each individual publication were specifically and individually indicated to be incorporated by reference as though fully set forth.

1. At least one chemical entity chosen from compounds of Formula (Ia): 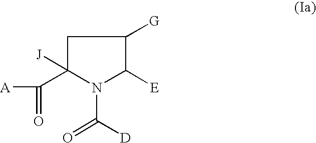
wherein:
A represents hydroxy;
D represents 4-tert-butyl-3-methoxyphenyl;
E represents 1,3-thiazol-2-yl or 5-methyl-1,3-thiazol-2-yl;
G represents methoxymethyl;
J represents 1,3-thiazol-2-ylmethyl, 1,3-thiazol-4-ylmethyl, 1,2-thiazol-3-ylmethyl, or 1H-pyrazol-1-ylmethyl;
and salts solvates and esters thereof; provided that when A is esterified to form —OR where R is selected from straight or branched chain alkyl, aralkyl, aryloxyalkyl, or aryl, then R is other than tert-butyl.

2. At least one chemical entity chosen from compounds of Formula (Ia) selected from the group consisting of:
rel-(2R,4S,5R)-1-(3-Methoxy-4-tert-butylbenzoyl)4-(methoxymethyl)-5-(5-methyl-1,3-thiazol-2-yl)-2-(1,3-thiazol-4-ylmethyl)pyrrolidine-2-carboxylic acid;
rel-(2R,4S ,5R)-1-(3-Methoxy-4-tert-butylbenzoyl)-4-(methoxymethyl)-5-(1,3-thiazol-2-yl)-2-(1,3-thiazol-4-ylmethyl)pyrrolidine-2-carboxylic acid;
rel-(2R,4S,5R)-1-(3-Methoxy-4-tert-butylbenzoyl)-4-(methoxymethyl)-5-(1,3-thiazol-2-yl)-2-(1,2-thiazol-3-ylmethyl)pyrrolidine-2-carboxylic acid;
rel-(2R,4S,5R)-1-(3-Methoxy-4-tert-butylbenzoyl)-4-(methoxymethyl)-2-(l H-pyrazol-1-ylmethyl)-5-(1,3-thiazol-2-yl)pyrrolidine-2-carboxylic acid;
rel-(2R,4S,5R)-1-(3-Methoxy-4-tert-butylbenzoyl)-4-(methoxymethyl)-5-(5-methyl-1,3-thiazol-2-yl)-2-(1,3-thiazol-2-ylmethyl)pyrrolidine-2-carboxylic acid;
rel-(2R,4S,5R)-1-(3-Methoxy-4-tert-butylbenzoyl)-4-(methoxymethyl)-5-(5-methyl-1,3-thiazol-2-yl)-2-(1,2-thiazol-3-ylmethyl)pyrrolidine-2-carboxylic acid; and
rel-(2R,4S,5R)-1-(3-Methoxy-4-tert-butylbenzoyl)-4-(methoxymethyl)-5-(5-methyl-1,3-thiazol-2-yl)-2-(1H-pyrazol-1-ylmethyl)pyrrolidine-2-carboxylic acid;
and salts, solvates and esters and individual enantiomers thereof.

3. A method of treating or preventing viral infection which comprises administering to a subject in need thereof, an effective amount of at least one chemical entity chosen from compounds of Formula (Ia) and salts, solvates and esters thereof as claimed in claim 1.

4. A method as claimed in claim 3 wherein the viral infection is HCV.

5. A method as claimed in claim 3 in which the chemical entity is administered in an oral dosage form.

6. (canceled)

7. (canceled)

8. (canceled)

9. (canceled)

10. (canceled)

11. A pharmaceutical formulation comprising at least one chemical entity chosen from compounds of Formula (Ia) and salts, solvates and esters thereof as claimed in claim 1 in conjunction with at least one pharmaceutically acceptable diluent or carrier.

12. A process for the preparation of a compound of Formula (Ia) as defined in claim 1 comprising deprotection of a compound of Formula (II) 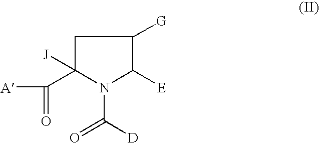
in which A′ is a protected hydroxy group, for example an alkoxy, benzyloxy or silyloxy,
D represents 4-tert-butyl-3-methoxyphenyl; E represents 1,3-thiazol-2-yl or 5-methyl-1,3-thiazol-2-yl; G represents methoxymethyl; and J represents 1,3-thiazol-2-ylmethyl, 1,3-thiazol-4-ylmethyl, 1,2-thiazol-3-ylmethyl, or 1H-pyrazol-1-ylmethyl.

13. (2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid or a pharmaceutically acceptable salt thereof.

14. A pharmaceutical formulation comprising (2R,4S,5R)-1-(3-Methoxy-4-tert-butylbenzoyl)4-(methoxymethyl)-2-(1H-pyrazol-1-ylmethyl)-5-(1,3-thiazol-2-yl)pyrrolidine-2-carboxylic acid or a pharmaceutically acceptable salt thereof in conjunction with at least one pharmaceutically acceptable diluent or carrier.

15. The pharmaceutical formulation according to claim 14 in the form of a tablet or capsule.

16. The pharmaceutical formulation according to claim 14 in the form of a solution or suspension.

17. A method of treating or preventing a hepatitis C infection which comprises administering to a subject in need thereof, an effective amount of (2R,4S,5R)-1-(3-Methoxy-4-tert-butylbenzoyl)-4-(methoxymethyl)-2-(1H-pyrazol-1-ylmethyl)-5-(1,3-thiazol-2-yl)pyrrolidine-2-carboxylic acid or a pharmaceutically acceptable salt thereof.
